(19) United States
(12) Patent Application Publication
(10) Pub. No.: US 2025/0043282 A1
(43) Pub. Date: Feb. 6, 2025

(54) ATTENUATED ORTHOMYXOVIRUS-BASED VECTOR PLATFORM

(71) Applicant: NEW YORK UNIVERSITY, New York, NY (US)

(72) Inventor: Benjamin tenOever, New York, NY (US)

(73) Assignee: NEW YORK UNIVERSITY, New York, NY (US)

(21) Appl. No.: 18/737,030

(22) Filed: Jun. 7, 2024

Related U.S. Application Data
(60) Provisional application No. 63/472,205, filed on Jun. 9, 2023.

(51) Int. Cl. C12N 15/113; A61K 48/00; C12N 15/86

(52) U.S. Cl. CPC C12N 15/113; A61K 48/0058; C12N 15/86; C12N 2310/14; C12N 2760/16143; C12N 2760/16152; C12N 2760/16162

(57) ABSTRACT

The present application provides an attenuated orthomyxovirus-based (e.g., influenza A virus (IAV)-based) replicon vector, which optionally encodes a biological cargo, and in which the one or more envelope glycoprotein transcripts are engineered to comprise one or more miRNA Silencing Elements (MSEs) such that the one or more vector envelope glycoproteins are expressed in a first cell type or species, but are not expressed in a second cell type or species, wherein the introduction of the attenuated replicon vector into the second cell type or species results in a sustained production of the biological cargo in the absence of vector propagation and cell death and/or toxicity. In other aspects, the present application is directed to the attenuated replicon vector production and its use for sustained production of biological cargo in vivo and prevention and treatment of diseases.

### CROSS-REFERENCE TO RELATED APPLICATIONS

This application claims priority to U.S. Provisional Application No. 63/472,205, filed Jun. 9, 2023, the disclosure of which is herein incorporated by reference in its entirety.

### STATEMENT REGARDING FEDERALLY SPONSORED RESEARCH OR DEVELOPMENT

This invention was made with government support under HR0011-21-2-0018 awarded by the Defense Advanced Research Projects Agency. The government has certain rights in the invention.

### SEQUENCE LISTING

The instant application contains a Sequence Listing which has been submitted electronically in XML file format and is hereby incorporated by reference in its entirety. Said XML copy, created on Aug. 23, 2024, is named 243735_000335_SL.xml and is 5,437 bytes in size.

### TECHNICAL FIELD

The present disclosure, in some aspects, is directed to an attenuated orthomyxovirus-based (e.g., influenza A virus (IAV)-based) replicon vector, which optionally encodes a biological cargo, and in which the one or more envelope glycoprotein transcripts are engineered to comprise one or more miRNA Silencing Elements (MSEs) such that the one or more vector envelope glycoproteins are expressed in a first cell type or species, but are not expressed in a second cell type or species, wherein the introduction of the attenuated replicon vector into the second cell type or species results in a sustained production of the biological cargo in the absence of vector propagation and cell death and/or toxicity. In other aspects, the present disclosure is directed to the attenuated replicon vector production and its use for sustained production of biological cargo in vivo and prevention and treatment of diseases.

### BACKGROUND

The field of gene and cell therapy has made many significant advances as it relates to treating diseases for which no other medical interventions exist. The therapeutic process comprises the introduction of function-altering, genetic material to a specific cell type of interest. To achieve this, the community generally exploits virus biology to engineer delivery platforms, which now comprise a class of therapeutics referred to as virotherapies (Dunbar, C. E. et al. (2018) Science 359). At present, the most clinically advanced virotherapies are based on adenovirus (AdV), lentivirus, and adeno-associated virus (AAV) biology, the latter of which is presently dominating the field (see, e.g., Bulcha, J. T. et al. (2021) Signal Transduct Target Ther 6, 53; Nathwani, A. C. et al. (2011) N Engl J Med 365, 2357-2365; Manno, C. S. et al. (2006) Nat Med 12, 342-347; Varnavski, A. N. et al. (2002) J Virol 76, 5711-5719; Frahm, N. et al. (2012) J Clin Invest 122, 359-367). Despite the many successes these vectors have achieved, further development of these existing platforms and the generation of alternative designs are still required to address the countless correctable diseases where delivery challenges remain insurmountable.

Influenza A virus (IAV) has several features that make it an attractive virotherapy platform. First, unlike the aforementioned vectors, IAV does not have a DNA phase during its replication cycle, and is thus unable to integrate into the host genome where it could cause unintentional interruption or deregulation of important host genes (Banasik, M. B. and McCray, P. B., Jr. (2010) Gene Ther 17, 150-157). Second, IAV biology has been intensely studied, materializing in countless tools and reagents to engineer desired genetic circuits, akin to what the study of HIV did for the lentiviral platform. Third, while seroprevalence for IAV in the human population is high, the vast global reservoir of IAV strains provides countless designs to circumvent this problem. Fourth, as an IAV-based platform has already received FDA-approval in the form of an intranasal, replication competent viral vaccine, commercialization and safety profiles are already in place to enable and support clinical development. Lastly, IA Vis an attractive vector for exogenous gene delivery as its biology does not permit homologous recombination, providing genetic stability and predictable biological behavior of a given design.

Despite the aforementioned attributes, utilizing IAV as a virotherapy platform is inherently limited by the duration of expression that can be achieved in vivo. IAV biology and transgene expression in target cells is relatively transient as these processes culminate in cell death and/or immune clearance. For example, a study in which IAV was utilized to deliver Cre-recombinase to Cre-dependent tdTomato reporter mice found that while ˜5.9% of pulmonary cells showed evidence of successful delivery ten days post administration, by twenty-one days, this population was reduced to 1.7% (Heaton, N. S. et al. (2014) J Exp Med 211, 1707-1714). Efforts to understand the clearance of IAV-treated cells have implicated the surface expression of the viral hemagglutinin (HA) and/or neuraminidase (NA) gene products which have been shown to both induce cell cytotoxicity and immune clearance, although other IAV transcripts also likely contribute (Flory, E. et al. (2000). J Biol Chem 275, 8307-8314). Thus, there is an unmet need for an improved IAV virotherapy platform.

### BRIEF SUMMARY

In one aspect, provided herein is a recombinant attenuated orthomyxovirus-based replicon (AOR) vector, wherein the AOR vector comprises vRNA segments collectively encoding orthomyxovirus polypeptides, or functional mutants or derivatives thereof, essential for AOR vector cell entry, cell exit and replication, wherein one or more orthomyxovirus envelope glycoproteins or functional mutants or derivatives thereof may be optionally replaced by one or more heterologous envelope glycoproteins which are capable of mediating AOR vector cell entry and/or exit, wherein one or more of the vRNA segments encoding the one or more orthomyxovirus envelope glycoproteins or functional mutants or derivatives thereof or the one or more heterologous envelope glycoproteins comprise one or more microRNA (miRNA) Silencing Elements (MSEs) such that said one or more orthomyxovirus envelope glycoproteins or functional mutants or derivatives thereof or said one or more heterologous envelope glycoproteins are not expressed or are expressed at a substantially lower level in a first cell type or species, but are expressed in a second cell type or species.

In some embodiments, the AOR vector is used for production of a biological cargo, wherein the AOR vector comprises vRNA segments collectively encoding the biological cargo, and orthomyxovirus polypeptides, or functional mutants or derivatives thereof, essential for AOR vector cell entry, cell exit and replication.

In some embodiments, the introduction of the AOR vector into the first cell type or species results in a sustained production of the biological cargo in the absence of the AOR envelope glycoproteins, resulting in self-amplification of the RNA.

In some embodiments, each of said one or more MSEs is targeted by a miRNA expressed in the first cell type or species, wherein said miRNA is not expressed or is expressed at a substantially lower level in the second cell type or species. In some embodiments, said miRNA is not expressed in the second cell type or species.

In some embodiments, said one or more MSEs are located within 3′ untranslated region (3′UTR) of vRNA segment(s) encoding the one or more orthomyxovirus envelope glycoproteins or functional mutants or derivatives thereof, or heterologous envelope glycoproteins.

As incorporation of the MSE into the 3′UTR can disrupt the packaging material of the vRNA segment, the packaging material may be further duplicated 3′ of the MSE. Packaging sequences at the 5′ end of the vRNA, where the MSE is incorporated, vary between both segments and strains but in general comprise 150, 100, 80, 125, 145, 190, 240, and 60 for segments 1, 2, 3, 4, 5, 6, 7, and 8, respectively. Because the vRNAs are negative sense, the start codon (AUG) is on the 3′ end of the vRNA segment. For this same reason, the 3′UTR of a given transcript is actually found on the 5′ end of the vRNA segment.

In some embodiments, said one or more MSEs are located within 5′ untranslated region (5′UTR) of vRNA segment(s) encoding the one or more orthomyxovirus envelope glycoproteins or functional mutants or derivatives thereof, or heterologous envelope glycoproteins.

In some embodiments, additional vRNA segment(s) encoding e.g., matrix proteins and/or polymerase subunits, may comprise one or more MSEs such that encoded proteins are not expressed or are expressed at a substantially lower level in a first cell type or species, but are expressed in a second cell type or species.

In some embodiments, the orthomyxovirus is an influenza virus. In some embodiments, the orthomyxovirus belongs to the influenza A, B, C, or D virus, Isavirus, or Thogotovirus genera.

In some embodiments, the biological cargo is an antigen, a biologically active polypeptide, a non-coding RNA (ncRNA), a marker polypeptide, one or more components of a gene editing system, or a Cre recombinase. In some embodiments, the biologically active polypeptide is a cytokine.

In some embodiments, when administered to a subject of the first species or comprising the first cell type, the AOR vector induces a substantially lower immune response as compared to a corresponding orthomyxovirus-based vector without the MSEs.

In some embodiments, the immune response is measured by i) determining the level of IgG and/or IgM to the one or more orthomyxovirus envelope glycoproteins or functional mutants or derivatives thereof or the one or more heterologous envelope glycoproteins, and/or ii) measuring anti-AOR vector T cell responses.

In one aspect, provided herein is a pharmaceutical composition comprising the AOR vector described herein and a pharmaceutically acceptable carrier or excipient.

In one aspect, provided herein is a method for producing a biological cargo in cells of a subject in need thereof, the method comprising administering to the subject an effective amount of the AOR vector described herein or the pharmaceutical composition described herein, wherein the subject belongs to the first species or the cells belong to the first cell type.

In one aspect, provided herein is a method for preventing or treating a disease in a subject in need thereof, the method comprising administering to the subject an effective amount of the AOR vector described herein or the pharmaceutical composition described herein, wherein the subject belongs to the first species. In some embodiments, the disease is an infection, an immune disease, a cancer, a disease treatable by an enzyme replacement therapy, or a disease treatable by gene editing.

In some embodiments, the AOR vector or pharmaceutical composition is administered intranasally, intravenously, intramuscularly, subcutaneously, intradermally, intrathecally, intraocularly, intra-arterially, intrapleurally, or intratumorally.

In some embodiments, the subject is human.

In some embodiments, the subject is a veterinary animal.

In one aspect, provided herein is a method of producing the AOR vector described herein, the method comprising introducing into a cell of the second cell type or species one or more polynucleotides encoding said vRNA segments of the AOR vector and incubating the cell under conditions suitable for AOR vector propagation.

In one aspect, the present disclosure provides an attenuated influenza A virus (IAV)-based replicon (AIR) vector, optionally for the production of a biological cargo. The compositions and methods provided herein address an inherent limitation of IAV clearance to enable the use of IAV as a virotherapy platform.

Thus, in some aspects, provided herein is a an attenuated influenza A virus (IAV)-based replicon (AIR) vector, wherein the AIR vector comprises IAV viral RNA (vRNA) segments (e.g., 7 or 8) collectively encoding polypeptides, or functional mutants or derivatives thereof, of the IAV genome essential for IAV vector cell entry, cell exit and replication, wherein at least one IAV vRNA segment of the IAV vRNA segments encodes a modulation moiety configured such that a polypeptide of the polypeptides of the IAV genome is not expressed or is inhibited in a first cellular environment, and wherein the target polypeptide is expressed and substantially functional in a second cellular environment. In some embodiments, a second polypeptide of the polypeptides encoded by the IAV genome is not expressed or is inhibited in the first cellular environment, and wherein the second polypeptide is expressed and substantially functional in the second cellular environment.

In some embodiments, the polypeptide(s) that are modulated by the modulation moiety are IAV hemagglutinin (HA) and/or neuraminidase (NA) or functional mutants or derivatives thereof. In some embodiments, IAV hemagglutinin (HA) and/or neuraminidase (NA) or functional mutants or derivatives thereof may be optionally replaced by one or more heterologous envelope glycoproteins which are capable of mediating AIR vector cell entry and/or exit,

In some embodiments, the modulation moiety is a microRNA (miRNA) Silencing Element (MSE) on an IAV mRNA produced from the least one IAV vRNA segment, wherein the IAV mRNA encodes the polypeptide, and wherein the MSE is targeted by a miRNA in the first cellular environment. In some embodiments, the MSE is within an untranslated region (UTR) of the IAV mRNA. In some embodiments, the MSE is located within a 5′ UTR of the IAV mRNA. In some embodiments, the MSE is located within a 3′ UTR of the IAV mRNA. In some embodiments, the MSE is within the open reading frame (ORF) of the IAV mRNA. In some embodiments, the miRNA silences expression of the polypeptide in the first cellular environment. In some embodiments, expression of the miRNA in the first cellular environment is at least 2 times higher than the expression of the miRNA in the second cellular environment. In some embodiments, the miRNA is not expressed in the second cellular environment. In some embodiments, the miRNA is from a vertebrate. In some embodiments, the miRNA is not present in the allantois of a fertilized egg. In some embodiments, the miRNA is selected from mir-21, mir-31, mir-192, mir-29a, mir-29b, mir-125a, mir-320a, mir-151, mir-1974, mir-331, mir-106b, mir-224, let-7e, mir-574, mir-320c-1, mir-215, mir-92b, mir-361, mir-378, mir-424, mir-423, mir-320b, mir-339, mir-1259, mir-99b, mir-28, mir-26b, mir-194-2, mir-183, mir-582, mir-1274a, mir-342, mir-1249, mir-1307, mir-494, mir-1180, mir-542, mir-452, mir-376c, mir-374b, mir-409, mir-194-1, mir-197, mir-34b, mir-152, let-7d, mir-345, mir-362, mir-505, mir-421, mir-487b, mir-625, mir-132, mir-503, mir-181c, mir-376a-1, mir-1280, mir-376a-2, mir-652, miR-124, miR-142, miR-1, miR-122, miR-200, miR-133a, miR-375, miR-143, miR-145, and miR-184, let-7a, miR-206, miR-155, miR-208, miR-499, miR-192, miR-7, miR-9, miR-223, miR-150, miR-137, miR-134, or any combination thereof. In some embodiments, the miRNA is selected from miR-21, miR-31, miR-192, miR-93, miR-29b, or any combination thereof.

In some embodiments, the modulation moiety is an inhibitory domain fused to the polypeptide, wherein the inhibitory domain comprises a protease motif cleavable by a protease, wherein the protease is not expressed or expressed at a substantially lower level in the first cellular environment, and wherein the protease is expressed in the second cellular environment. In some embodiments, the inhibitory domain is encoded within the ORF of an IAV mRNA produced from the at least one vRNA segment, wherein the IAV mRNA encodes the polypeptide. In some embodiments, expression of the protease in the second cellular environment is at least 2 times higher than expression of the protease in the first cellular environment. In some embodiments, cleavage of the protease motif by the protease in the second cellular environment releases the inhibitory domain from the polypeptide. In some embodiments, the protease motif is a C1s motif, an elastase motif, a cathepsid motif, a metalloproteinase, a trypsin motif, or a plasminogen activator motif. In some embodiments, the protease is C1s, elastase, cathepsin B/D/G/L/S, trypsin, or plasminogen. In some embodiments, the inhibitory domain comprises a polypeptide that inhibits function of the target polypeptide of the IAV genome. In some embodiments, the inhibitory domain comprises a negatively charged polypeptide. In some embodiments, the inhibitory domain comprises a positively charged polypeptide.

In some embodiments, the modulation moiety is a splicing motif comprising a 5′-splice site and a 3′-splice site on an IAV mRNA produced from the at least one IAV vRNA segment, wherein the IAV mRNA encodes the polypeptide, wherein a region between the 5′-splice site and a 3′-splice site is not spliced out of the splicing motif in the first cellular environment such that no functional polypeptide is expressed, and wherein the region between the 5′-splice site and a 3′-splice site is spliced out of the splicing motif in the second cellular environment such that a functional polypeptide is expressed. In some embodiments, the splicing motif comprises a sequence derived from the homothorax or Dcam endogenous gene from insects or a flowering locus C (FLC) gene in plants.

In some embodiments, the modulation moiety is an internal ribosome entry site (IRES) motif on an IAV mRNA produced from the at least one IAV vRNA segment, wherein the IAV mRNA encodes the polypeptide, wherein the IRES motif recruits a ribosome in the second cellular environment such that translation of the polypeptide can be initiated, and wherein, the IRES motif does not recruit a ribosome in the first cellular environment such that translation of the polypeptide cannot be initiated. In some embodiments, the IRES motif is immediately 5′ to a translational start site of the ORF encoding the polypeptide. In some embodiments, the IRES motif is from Tobacco Mosiac virus, Barley yellow dwarf virus, Turnip Crinkle virus, Cowpeae mosaic virus, tobacco etch virus, or potato virus A.

In some embodiments, the first cellular environment is a first cell type and the second cellular environment is a second cell type. In some embodiments, the first cellular environment is a first tissue and the second cellular environment is a second tissue. In some embodiments, the first tissue and the second tissue are from the same species. In some embodiments, the first cellular environment is a first organelle and the second cellular environment is a second organelle. In some embodiments, the first organelle and the second organelle are from the same species. In some embodiments, the first organelle and the second organelle are from the same cell. In some embodiments, the first organelle and the second organelle are from different cells.

In some embodiments, the first cellular environment is a first species and the second cellular environment is a second species. In some embodiments, the first species is human. In some embodiments, the second species is chicken, monkey, canine, insect, or plant. In some embodiments, the second cellular environment is an embryonated chicken egg. In some embodiments, the second cellular environment is selected from the group consisting of chicken fibroblasts DF1, Madin-Darby Canine Kidney (MCK) cells, African green monkey kidney cells (Vero), human PER-C6 cells. C6/36 mosquito cells, Nicotiana benthamiana, Arabidopsis thaliana, Medicago sativa, Zea mays, and Solanum tuberosum.

In some embodiments, the polypeptides encoded by the IAV genome are PB1, PB2, PA, HA, NP, NA, M1, M2, NS1, and NS2/NEP.

In some embodiments, the polypeptides encoded by the IAV genome are PB1, PB2, PA, HA, NP, NA, M1, M2, and NS2/NEP.

In some embodiments, the AIR vector comprises eight (8) IAV vRNA segments, and wherein the eight IAV vRNA segments collectively encode PB1, PB2, PA, HA, NP, NA, M1, M2, NS1, and NS2/NEP. In some embodiments, the 8 IAV vRNA segments comprise: (i) IAV vRNA segment 1, wherein IAV vRNA segment 1 is about 2.3 kB in length; (ii) IAV vRNA segment 2, wherein IAV vRNA segment 2 is about 2.3 kB in length; (iii) IAV vRNA segment 3, wherein IAV vRNA segment 3 is about 2.1 kB in length; (iv) IAV vRNA segment 4, wherein IAV vRNA segment 4 is about 1.5 KB in length; (v) IAV vRNA segment 5, wherein IAV vRNA segment 5 is about 1.4 kB in length; (vi) IAV vRNA segment 6, wherein IAV vRNA segment 6 is about 1.0 kB in length for NA; (vii) IAV vRNA segment 7, wherein IAV vRNA segment 7 is about 0.9 kB in length; and (viii) IAV vRNA segment 8, wherein IAV vRNA segment 8 is about 0.8 KB in length.

In some embodiments, one or more of the IAV vRNA segments encodes a biological cargo. In some embodiments, the biological cargo is not encoded by IAV vRNA segment 7. In some embodiments, the biological cargo is encoded by: (i) IAV vRNA segment 1, wherein the sequence encoding the biological cargo comprises up to about 1,500 nucleotides; (ii) IAV vRNA segment 2, wherein the sequence encoding the biological cargo comprises up to about 1,000 nucleotides; (iii) IAV vRNA segment 3, wherein the sequence encoding the biological cargo comprises up to about 2,300 nucleotides; (iv) IAV vRNA segment 4, wherein the sequence encoding the biological cargo comprises up to about 700 nucleotides; (v) IAV vRNA segment 5, wherein the sequence encoding the biological cargo comprises up to about 900 nucleotides; (vi) IAV vRNA segment 6, wherein the sequence encoding the biological cargo comprises up to about 1,000 nucleotides; and/or, (vii) IAV vRNA segment 8, wherein the sequence encoding the biological cargo comprises up to about 1,000 nucleotides. In some embodiments, the sequence encoding the biological cargo is an antigen, a biologically active polypeptide (e.g., cytokine), a non-coding RNA (ncRNA), a marker polypeptide (e.g., green fluorescent protein (GFP)), one or more components of a gene editing system, or a Cre recombinase. In some embodiments, the biological cargo is an antigen, and wherein the antigen induces immunity against a pathogen in the first cellular environment. In some embodiments, the biological cargo is a biologically active polypeptide, and wherein the biologically active polypeptide replaces the function of a defective gene in the first cellular environment. In some embodiments, the at least 7 IAV vRNA segments comprise: (i) IAV vRNA segment 1 encoding PB2 and NS1; (ii) IAV vRNA segment 2 encoding PB1 and NS2; (iii) IAV vRNA segment 3 encoding PA; (iv) IAV vRNA segment 4 encoding HA; (v) IAV vRNA segment 5 encoding NP; (vi) IAV vRNA segment 6 encoding NA; (vii) IAV vRNA segment 7 encoding M1 and M2; and (viii) IAV vRNA segment 8 encoding a biological cargo.

In some embodiments, the target polypeptide is HA.

In some embodiments, the target polypeptide is NA.

In some embodiments, the target polypeptide is M1 and/or M2.

In some embodiments, the target polypeptide is PA.

In some embodiments, two or more polypeptides of the polypeptides of the IA V genome are not expressed or inhibited in the first cellular environment. In some embodiments, the two or more polypeptides comprise HA and NA. In some embodiments, the two or more polypeptides comprise HA, NA, M1 and/or M2.

In some embodiments, the AIR vector exhibits reduced inflammation by greater than about 80% in the first cellular environment compared to the second cellular environment. In some embodiments, inflammation is measured by i) IgG and IgM to a targeted IAV protein, or ii) monitoring the transcriptional host response.

In some embodiments, the AIR vector is not capable of propagating in the first cellular environment.

In some embodiments, the target polypeptide is not expressed or is inhibited in the first cellular environment for between about 2 to about 14 days after vector administration.

In some aspects, provided herein is a pharmaceutical composition comprising the AIR vector of any of the preceding embodiments and a pharmaceutically acceptable carrier or excipient.

In some aspects, provided herein is a method of preventing or treating a disease in a subject, the method comprising administering an effective amount of the AIR vector of any of the preceding embodiments or the pharmaceutical composition of the preceding embodiment to the subject, wherein the subject comprises the first cellular environment. In some embodiments, the disease is an infection (e.g., viral infection), an immune disease, a cancer, a disease treatable by an enzyme replacement therapy, or a disease treatable by gene editing. In some embodiments, the viral infection is from an IAV. In some embodiments, the disease benefits from a targeted therapy.

In some aspects, provided herein is a method of delivering a gene to a subject, the method comprising administering the AIR vector of any of the preceding embodiments, or a pharmaceutical composition thereof, to the subject, wherein the subject comprises the first cellular environment.

In some aspects, provided herein is a method of delivering a gene to a cell, the method comprising administering the AIR vector of any of the preceding embodiments, or a pharmaceutical composition thereof, to the cell, wherein the cell comprises the first cellular environment.

In some aspects, provided herein is a method of producing an IAV, the method comprising introducing into a cell comprising the second cellular environment one or more polynucleotides encoding said vRNA segments of the AIR vector of any of the preceding embodiments and incubating the cell under conditions suitable for AIR vector propagation.

In one aspect, provided herein is a recombinant attenuated influenza A virus (IAV)-based replicon (AIR) vector for production of a biological cargo, wherein the AIR vector comprises vRNA segments collectively encoding the biological cargo and IAV polypeptides, or functional mutants or derivatives thereof, essential for AOR vector cell entry, cell exit and replication, wherein IAV hemagglutinin (HA) and/or neuraminidase (NA) or functional mutants or derivatives thereof may be optionally replaced by one or more heterologous envelope glycoproteins which are capable of mediating AIR vector cell entry and/or exit, wherein the vRNA segment(s) encoding HA and/or NA or the one or more functional mutants or derivatives thereof or the one or more heterologous envelope glycoproteins comprise one or more microRNA (miRNA) Silencing Elements (MSEs) such that said HA and/or NA or the one or more functional mutants or derivatives thereof or the one or more heterologous envelope glycoproteins are not expressed or are expressed at a substantially lower level in a first cell type or species, but are expressed in a second cell type or species.

In some embodiments, the AIR vector comprises at least seven vRNA segments collectively encoding the biological cargo and IAV polypeptides, or functional mutants or derivatives thereof, essential for AOR vector cell entry, cell exit and replication.

In some embodiments, the AIR vector comprises eight vRNA segments collectively encoding the biological cargo and IAV polypeptides, or functional mutants or derivatives thereof, essential for AOR vector cell entry, cell exit and replication.

In some embodiments, (i) the vRNA segment(s) encoding IAV matrix protein 1 (M1) and matrix protein 2 (M2), or functional mutants or derivatives thereof; and/or (ii) the vRNA segment encoding IAV polymerase PA subunit, or a functional mutant or derivative thereof, comprise one or more microRNA (miRNA) Silencing Elements (MSEs) such that said protein(s) or the one or more functional mutants or derivatives thereof are not expressed or are expressed at a substantially lower level in a first cell type or species, but are expressed in a second cell type or species.

In some embodiments, said one or more MSEs is targeted by a miRNA expressed in the first cell type or species, wherein said miRNA is not expressed or is expressed at a substantially lower level in the second cell type or species. In some embodiments, said miRNA is not expressed in the second cell type or species.

In some embodiments, the second species is chicken allantois and the first species is human, and said one or more MSEs are targeted by miR-21, miR-31, miR-192, miR-93, miR-29b, or any combination thereof.

In some embodiments, the vRNA segment(s) encoding HA and/or NA or the one or more functional mutants or derivatives thereof or the one or more heterologous envelope glycoproteins, the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof, comprises two or more, three or more, or four or more different MSEs which are targeted by miR-21, miR-31, miR-192, miR-93, or miR-29b, or a combination thereof.

In some embodiments, the vRNA segment(s) encoding HA and/or NA or the one or more functional mutants or derivatives thereof or the one or more heterologous envelope glycoproteins, the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof, comprises five MSEs which are targeted by miR-21, miR-31, miR-192, miR-93, and miR-29b, positioned in any order.

In some embodiments, (i) the second species is chicken allantois, monkey, canine, insect, or plant, and (ii) the first species is human.

In one aspect, provided herein is a recombinant attenuated influenza A virus (IAV)-based replicon (AIR) vector, wherein the AIR vector comprises vRNA segments collectively encoding IAV polypeptides, or functional mutants or derivatives thereof, essential for AOR vector cell entry, cell exit and replication, wherein IAV hemagglutinin (HA) and/or neuraminidase (NA) or functional mutants or derivatives thereof may be optionally replaced by one or more heterologous envelope glycoproteins which are capable of mediating AIR vector cell entry and/or exit, wherein the vRNA segment(s) encoding HA and/or NA or the one or more functional mutants or derivatives thereof or the one or more heterologous envelope glycoproteins comprise one or more microRNA (miRNA) Silencing Elements (MSEs) such that said HA and/or NA or the one or more functional mutants or derivatives thereof or the one or more heterologous envelope glycoproteins are not expressed or are expressed at a substantially lower level in a first cell type or species, but are expressed in a second cell type or species; and wherein said one or more MSEs are targeted by at least one of miR-21, miR-31, miR-192, miR-29b, or any combination thereof. In some embodiments, said one or more MSEs are further targeted by miR-93.

In some embodiments, the AIR vector comprises at least seven vRNA segments collectively encoding the IAV polypeptides, or functional mutants or derivatives thereof, essential for AOR vector cell entry, cell exit and replication.

In some embodiments, (i) the vRNA segment(s) encoding IAV matrix protein 1 (M1) and matrix protein 2 (M2), or functional mutants or derivatives thereof; and/or (ii) the vRNA segment encoding IAV polymerase PA subunit, or a functional mutant or derivative thereof, comprise one or more microRNA (miRNA) Silencing Elements (MSEs) such that said protein(s) or the one or more functional mutants or derivatives thereof are not expressed or are expressed at a substantially lower level in a first cell type or species, but are expressed in a second cell type or species; and wherein said one or more MSEs are targeted by at least one of miR-21, miR-31, miR-192, miR-29b, or any combination thereof. In some embodiments, said one or more MSEs are further targeted by miR-93.

In some embodiments, the vRNA segment(s) encoding HA and/or NA or the one or more functional mutants or derivatives thereof or the one or more heterologous envelope glycoproteins, the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof, comprises five MSEs which are targeted by miR-21, miR-31, miR-192, miR-93, and miR-29b, positioned in any order.

In some embodiments, the AIR vector comprises a sequence encoding a biological cargo. In some embodiments, the AIR vector comprises eight vRNA segments collectively encoding the biological cargo and IAV polypeptides, or functional mutants or derivatives thereof, essential for AOR vector cell entry, cell exit and replication.

In some embodiments, said one or more MSEs are located within 3′ untranslated region (3′UTR) of the vRNA segment(s) encoding HA and/or NA or the one or more functional mutants or derivatives thereof or the one or more heterologous envelope glycoproteins.

In some embodiments, said one or more MSEs are located within 5′ untranslated region (5′UTR) of the vRNA segment(s) encoding HA and/or NA or the one or more functional mutants or derivatives thereof or the one or more heterologous envelope glycoproteins.

In some embodiments, the vRNA segment(s) encoding HA and NA comprise(s) one or more MSEs such that said HA and NA are expressed in a second cell type or species, but are not expressed or are expressed at a substantially lower level in a first cell type or species.

In some embodiments, said one or more MSEs are located within 3′ untranslated region (3′UTR) of the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof.

In some embodiments, said one or more MSEs are located within 5′ untranslated region (5′UTR) of the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof.

In some embodiments, (i) the second species is chicken allantois, and (ii) the first species is human.

In some embodiments, (i) the second cell type is an embryonated chicken egg, and (ii) the first cell type is a human cell.

In some embodiments, the vRNA segments collectively encode PB1, PB2, PA, HA, NP, NA, M1, M2, NS1, and NS2/NEP or functional mutants or derivatives thereof.

In some embodiments, the vRNA segments collectively encode PB1, PB2, PA, HA, NP, NA, M1, M2, and NS2/NEP or functional mutants or derivatives thereof.

In some embodiments, the functional mutants are temperature sensitive mutants.

In some embodiments, the temperature sensitive mutants are PB11195/2005 (K391E and/or A661T), PB11766 (E581G), PB2821 (N265S), NP146 (D34G), or any combination thereof.

In some embodiments, the heterologous envelope glycoprotein is a vesiculovirus G protein, thogotovirus G protein, or a functional mutant or derivative thereof.

In some embodiments, the vRNA segments collectively encode PB1, PB2, PA, thogotovirus G protein, NP, M1, M2, and NS2/NEP or functional mutants or derivatives thereof.

In some embodiments, the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or a functional mutant or derivative thereof;
and
(viii) vRNA segment 8 encoding the biological cargo;

wherein a) vRNA segment 1 further encodes NS1 or a functional mutant or derivative thereof, and vRNA segment 2 further encodes NS2 or a functional mutant or derivative thereof; b) vRNA segment 1 further encodes NS1 or a functional mutant or derivative thereof, and vRNA segment 3 further encodes NS2 or a functional mutant or derivative thereof; c) vRNA segment 2 further encodes NS1 or a functional mutant or derivative thereof, and vRNA segment 3 further encodes NS2 or a functional mutant or derivative thereof; d) vRNA segment 1 further encodes NS2 or a functional mutant or derivative thereof, and vRNA segment 2 further encodes NS1 or a functional mutant or derivative thereof; e) vRNA segment 1 further encodes NS2 or a functional mutant or derivative thereof, and vRNA segment 3 further encodes NS1 or a functional mutant or derivative thereof; or f) vRNA segment 2 further encodes NS2 or a functional mutant or derivative thereof, and vRNA segment 3 further encodes NS1 or a functional mutant or derivative thereof.

In some embodiments, the vRNA segments comprise:
(i) vRNA segment 1 encoding PB2 and NS1 or functional mutants or derivatives thereof;
(ii) vRNA segment 2 encoding PB1 and NS2 or functional mutants or derivatives thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or a functional mutant or derivative thereof; and
(viii) vRNA segment 8 encoding the biological cargo.

In some embodiments, the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding the biological cargo; and
(viii) vRNA segment 8 encoding NS1 and NS2/NEP or functional mutants or derivatives thereof;

wherein a) vRNA segment 1 further encodes M1 or a functional mutant or derivative thereof, and vRNA segment 2 further encodes M2 or a functional mutant or derivative thereof; b) vRNA segment 1 further encodes M1 or a functional mutant or derivative thereof, and vRNA segment 3 further encodes M2 or a functional mutant or derivative thereof; c) vRNA segment 2 further encodes M1 or a functional mutant or derivative thereof, and vRNA segment 3 further encodes M2 or a functional mutant or derivative thereof; d) vRNA segment 1 further encodes M2 or a functional mutant or derivative thereof, and vRNA segment 2 further encodes M1 or a functional mutant or derivative thereof; e) vRNA segment 1 further encodes M2 or a functional mutant or derivative thereof, and vRNA segment 3 further encodes M1 or a functional mutant or derivative thereof; or f) vRNA segment 2 further encodes M2 or a functional mutant or derivative thereof, and vRNA segment 3 further encodes M1 or a functional mutant or derivative thereof.

In some embodiments, the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or functional mutants or derivatives thereof; and
(viii) vRNA segment 8 encoding NS1 and NS2/NEP or functional mutants or derivatives thereof;

wherein vRNA segment 1, 2, or 3 further encodes the biological cargo.

In some embodiments, the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof, and the biological cargo;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or functional mutants or derivatives thereof; and
(viii) vRNA segment 8 encoding NS1 and NS2/NEP or functional mutants or derivatives thereof.

In some embodiments, the vRNA segments comprise:
(i) vRNA segment 1 encoding PB2 or a functional mutant or derivative thereof;
(ii) vRNA segment 2 encoding PB1 or functional mutants or derivatives thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or functional mutants or derivatives thereof;
and
(viii) vRNA segment 8 encoding NS1, the biological cargo, and NS2/NEP or functional mutants or derivatives thereof.

In some embodiments, the vRNA segments comprise:
(i) vRNA segment 1 encoding PB2 or a functional mutant or derivative thereof;
(ii) vRNA segment 2 encoding PB1 or functional mutants or derivatives thereof;
(iii) vRNA segment 3 encoding PA or a functional mutant or derivative thereof;
(iv) vRNA segment 4 encoding thogotovirus G protein or a functional mutant or derivative thereof;
(v) vRNA segment 5 encoding NP or a functional mutant or derivative thereof;
(vi) vRNA segment 6 encoding the biological cargo;
(vii) vRNA segment 7 encoding M1 and M2 or functional mutants or derivatives thereof; and
(viii) vRNA segment 8 encoding NS1 and NS2/NEP or functional mutants or derivatives thereof.

In some embodiments, the biological cargo is an antigen, a biologically active polypeptide, a non-coding RNA (ncRNA), a marker polypeptide, one or more components of a gene editing system, or a Cre recombinase.

In some embodiments, the introduction of the AIR vector into the first cell type or species results in a sustained production of the biological cargo in the absence of the AIR vector propagation and infected cell death and/or toxicity. In some embodiments, the sustained production of the biological cargo is for at least 7 days. In some embodiments, the sustained production of the biological cargo is for 7-10 days.

In some embodiments, when administered to a subject of the first species, the AIR vector induces a substantially lower immune response as compared to the corresponding IAV-based vector without the MSEs. In some embodiments, the immune response is measured by i) determining the level of IgG and/or IgM to HA and/or NA or the one or more functional mutants or derivatives thereof or the one or more heterologous envelope glycoproteins, and/or ii) measuring anti-AIR vector T cell responses.

In some embodiments, said one or more MSEs comprise one or more reverse complement sequences of miR-21 comprising the sequence: UAGCUUAUCAGACUGAUGUUGA (SEQ ID NO: 1), miR-31 comprising the sequence AGGCAAGAUGCUGGCAUAGCU (SEQ ID NO: 2), miR-192 comprising the sequence CUGACCUAUGAAUUGACAGCC (SEQ ID NO: 3), miR-93 comprising the sequence CAAAGUGCUGUUCGUGCAGGUAG (SEQ ID NO: 4), or miR-29b-3p: UAGCACCAUUUGAAAUCAGUGUU (SEQ ID NO: 5), or any combination thereof.

In some embodiments, said one or more MSEs comprise the reverse complement sequences of miR-21 comprising the sequence: UAGCUUAUCAGACUGAUGUUGA (SEQ ID NO: 1), miR-31 comprising the sequence AGGCAAGAUGCUGGCAUAGCU (SEQ ID NO: 2), miR-192 comprising the sequence CUGACCUAUGAAUUGACAGCC (SEQ ID NO: 3), miR-93 comprising the sequence CAAAGUGCUGUUCGUGCAGGUAG (SEQ ID NO: 4), and miR-29b-3p: UAGCACCAUUUGAAAUCAGUGUU (SEQ ID NO: 5), positioned in any order.

In one aspect, provided herein is a pharmaceutical composition comprising the AIR vector described herein and a pharmaceutically acceptable carrier or excipient.

In one aspect, provided herein is a method for producing a biological cargo in airway cells of a subject in need thereof, the method comprising administering to the subject an effective amount of the AIR vector described herein or the pharmaceutical composition described herein, wherein the subject belongs to the first species or comprises the first cell types.

In one aspect, provided herein is a method for preventing or treating a disease in a subject in need thereof, the method comprising administering to the subject an effective amount of the AIR vector described herein or the pharmaceutical composition described herein, wherein the subject belongs to the first species or comprises the first cell types. In some embodiments, the disease is an infection, an immune disease, a cancer, a disease treatable by an enzyme replacement therapy, or a disease treatable by gene editing.

In some embodiments, the AIR vector or pharmaceutical composition is administered intranasally, intravenously, intramuscularly, subcutaneously, intradermally, intrathecally, intraocularly, intra-arterially, intrapleurally, or intratumorally.

In some embodiments, the subject is human.

In some embodiments, the subject is a veterinary animal.

In one aspect, provided herein is a method of producing the AIR vector described herein, the method comprising introducing into a cell of the second cell type or species one or more polynucleotides encoding said vRNA segments of the AIR vector and incubating the cell under conditions suitable for AIR vector propagation.

### BRIEF DESCRIPTION OF THE DRAWINGS

The patent or application file contains positioned in any order at least one drawing executed in color. Copies of this patent or patent application publication with color drawing(s) will be provided by the Office upon request and payment of the necessary fee.

The drawings illustrate certain embodiments of the features and advantages of this disclosure. These embodiments are not intended to limit the scope of the appended claims in any manner.

FIGS. 1A-1D show an exemplary schematic of various mechanisms for generating an attenuated orthomyxovirus-based replicon (AOR) vector such as an influenza A virus (IAV)-based replicon (AIR) vector, in accordance with some embodiments. FIG. 1A shows an exemplary schematic of a cell-specific protease design in which a protease motif (circle structures) tethers an inhibitory domain to a desired protein of interest which must be removed to enable functionality of the protein. FIG. 1B shows an exemplary schematic of a species-specific internal ribosome entry site (IRES) which enables translation only in cells in which the ribosome recognizes the structure to engage translation. FIG. 1C shows an exemplary schematic of species-specific splicing in which 5′ and 3′ splice motifs are only recognized in certain species to produce the correctly spliced product. FIG. 1D shows an exemplary schematic microRNA (miRNA)-mediated targeted of a desired transcript which can be designed for selective silencing based on the target sequence and the specific expression of its cognate miRNA.

FIG. 2A shows an exemplary schematic of the AIR vector depicting the eight viral RNA (vRNA) segments of the AIR vector, including the grafting of NS1 and NS2 onto PB2 and PB1, respectively, in addition to the introduction of miRNA Silencing Elements (shown as *) in HA and NA, for delivery of a cargo (e.g., a Cre recombinase).

FIG. 2B shows virus titers of IAV (A/California/04/2009) and the AIR vector shown in FIG. 2A in eggs, A549, and MDCK cells.

FIG. 2C shows an immunoblot of MDCK cells treated with PBS, infected with IAV, or administered the AIR vector. Blots were probed to demonstrate levels of IAV-derived HA, NA, NP and the host protein GAPDH.

FIG. 2D shows an ELISA of IgG titers for HA, NA, and NP from the serum of mice treated twice over a three-week period with PBS, IAV, or the AIR vector.

FIG. 3A shows results of RT-qPCR from the airways of mice treated with either PBS, IAV (A/California/04/2009), or the AIR vector and euthanized at 3-, 5-, 7-, and 10-days post administration.

FIG. 3B shows the induction of interferon beta (IFNβ) from the airways of mice treated with either PBS, IAV (A/California/04/2009), or the AIR vector and euthanized at 3-, 5-, 7-, and 10-days post administration.

FIG. 3C shows virus titers of IAV (A/California/04/2009) from the denoted organs from mice treated with either PBS, IAV (A/California/04/2009), or the AIR vector and euthanized at 3-, 5-, 7-, and 10-days post administration. Data is presented as plaque forming units per milliliter (pfu/mL).

FIG. 4A shows virus titers of IAV (A/California/04/2009) from the nasal wash of ferrets treated with PBS, infected with IAV (A/California/04/2009), or administered the AIR vector. Data is presented as plaque forming units per milliliter (pfu/mL).

FIG. 4B shows results of RT-qPCR from the nasal wash of ferrets treated with PBS, infected with IAV (A/California/04/2009), or administered the AIR vector to define the level of NP as compared to host derived beta actin (ACTB).

FIG. 4C shows results of immunohistochemistry of ferret trachea on day five from ferrets treated with PBS, infected with IAV (A/California/04/2009), or administered the AIR vector and euthanized at 5-days post administration. Top and bottom panels denote NP and NA staining, respectively.

FIG. 5A shows exemplary cryopreserved trachea from Ai9 mice treated with AIR alone or an AIR vector encoding Cre-recombinase. The signals denote tdTomato expression and DAPI-stained cell nuclei.

FIG. 5B shows exemplary cryopreserved lung from Ai9 mice treated with AIR alone or an AIR vector encoding Cre-recombinase. The signals denote tdTomato expression and DAPI-stained cell nuclei.

FIG. 6 shows an exemplary Influenza A virus (IAV), comprising a segmented genome (8 viral RNAs, i.e., vRNAs) in negative polarity bound by NP and the RdRp to form ribonucleoprotein complexes.

FIGS. 7A-7D show schematics of different miRNA-targeting strategies to develop an AIR vector delivery system. FIG. 7A shows a design similar to FIG. 2A. FIG. 7B shows an alternative design where segment 8 is unchanged from the wild-type version, and the cargo of interest is inserted in segment 3 encoding PA using a P2A site. miRNA Silencing Elements (shown as *) are introduced to the segments encoding HA and NA. FIG. 7C shows another alternative design where the sequence encoding a cargo of interest is inserted in segment 8 between the NS1 and NS2 ORFs. This design leverages the splicing activity of IAV. Normally the NS2 ORF overlaps with NS1, but it has been demonstrated that these ORFs can be separated to create available genetic space in between (Varble et al. PNAS. Jun. 7, 2010; 107 (25) 11519-11524). This space can be used to express a cargo of interest (e.g., polypeptide cargo) using an NS1-P2A strategy. Alternatively, a non-coding RNA cargo can be produced by placing the desired RNA after the stop codon of NS1. This allows the ncRNA to be liberated as a lariat when NS2 is synthesized. miRNA Silencing Elements (shown as *) are introduced to the segments encoding HA and NA. FIG. 7D shows another alternative design where Thogotovirus G protein is incorporated in place of HA and also targeted by the miRNAs (shown as *). As Thogotovirus G protein is sufficient to mediate viral cell entry and egress, this frees segment 6 (normally encoding NA) to be used to encode a cargo of interest.

FIGS. 8A-8B demonstrate the successful delivery of a cargo using two AIR vectors. FIG. 8A shows a schematic of an AIR design where segment 3 is used to encode a cargo (Cre recombinase) and miRNA Silencing Elements (shown as *) are introduced to the segments encoding HA and NA. FIG. 8B shows a schematic of an AIR design similar to FIG. 8A but the miRNA Silencing Elements (shown as *) are additionally introduced into the segment encoding M1/M2. Results from a reporter assay using human lung epithelial cells (A549) Cre-reporter cells are shown below each schematic. The panels show GFP expression upon delivery of the Cre recombinase at the indicated multiplicity of infections (MOI). The images were generated 72 hours post AIR vector administration.

FIGS. 9A-9C show in vivo data from mice treated with 10\hat{ }4 pfu of the HA/NA miRNA targeted AIR vector where different cargos are encoded in segment 3. The cargos included (FIG. 9A) interleukin 1 receptor antagonist (ILIRN), (FIG. 9B) interleukin 10 (IL10), and (FIG. 9C) transforming growth factor beta (TGF-B). The graphs below each schematic show expression of the respective cargos as measured by RT-qPCR from lung tissues of the treated mice at the indicated days post infection (DPI).

FIGS. 10A-10D show in vitro data from A549 cells treated with the HA/NA miRNA targeted AIR vector (MOI=1) where different cargos (cytokines) are encoded in segment 3 as described in FIG. 9. FIG. 10A shows a schematic of the study design. FIGS. 10B-10D show results from an enzyme linked immunosorbent assay (ELISA) measuring levels of the respective cytokines, (FIG. 10B) ILIRN, (FIG. 10C) IL10, and (FIG. 10D) TGF-β, in the supernatants collected from the A549 cells at 4, 8, 12, 24 hours post AIR vector treatment.

FIG. 11 shows RNA-Seq data from mice treated with HA/NA miRNA targeted AIR vector alone (HANA), as well as the same vector but expressing ILIRN (HANA-ILIRN), IL10 (HANA-IL10), or TGF-β (HANA-TGF-β) as described FIGS. 9 and 10. To assess functionality of HANA-delivered cytokines in vivo, the vectors were administered to mice one day following TLR7 ligand activation with R848 (intranasal). 48 hours post R848 treatment, lungs were isolated and assessed by bulk RNA sequencing. The heat map denotes differentially stimulated genes across samples and, on the right, are the GO annotations denoting the significant pathways impacted by these treatments.

### DETAILED DESCRIPTION

In some aspects, provided herein is an attenuated influenza A virus (IAV)-based replicon (AIR) vector. In some embodiments, the AIR vector comprises at least 7 IAV viral RNA (vRNA) segments collectively encoding polypeptides of the IAV genome, or functional mutants or derivatives thereof, wherein at least one IAV vRNA segment of the at least seven IAV vRNA segments encodes a modulation moiety configured such that a target polypeptide of the polypeptides of the IAV genome is not expressed or is inhibited in a first cellular environment, and wherein the target polypeptide is expressed and substantially functional in a second cellular environment. In some embodiments, the modulation moiety is a miRNA Silencing Element (MSE), an inhibitory domain, a splicing motif, an IRES motif, or other motifs which regulatory control results in conditional production of a desired product. Also provided herein are methods of use of the novel AIR vector.

The present disclosure is based, in part, on the inventors' unique insight that species—and/or cell-specific biological machinery may be exploited to generate constructs in which the translation of desired IAV transcripts is disrupted in target host populations while enabled in environments where vector propagation is desired. The AIR vector provided herein is a virotherapy-compatible platform that does not cause disease or result in extensive cell death either as a product of viral activity or immune engagement. Moreover, the engineered AIR vector is scalable, allowing manufacture at a clinical grade. The AIR vector may be used, for example, as a vaccine platform or to deliver a biological cargo (e.g., for gene therapy or for cell therapy).

In some embodiments, provided herein is an AIR vector, wherein the AIR vector comprises at least seven (7) IAV vRNA segments collectively encoding polypeptides, or functional mutants or derivatives thereof, of the IAV genome essential for AOR vector cell entry, cell exit and replication, wherein at least one IAV vRNA segment of the at least 7 IAV vRNA segments encodes a miRNA Silencing Element (MSE) configured such that a target polypeptide of the polypeptides of the IAV genome is not expressed or is inhibited in a first cellular environment, and wherein the target polypeptide is expressed and substantially functional in a second cellular environment. In some embodiments, the miRNA Silencing Element (MSE) is on an IAV mRNA produced from the least one IAV vRNA segment, the IAV mRNA encodes the target polypeptide, and the MSE is targeted by a miRNA in the first cellular environment. In some embodiments, the miRNA silences expression of the target polypeptide in the first cellular environment. In some embodiments, the miRNA is not expressed or is expressed at a substantially lower level in the second cellular environment. In some embodiments, the MSE is on the IAV mRNA produced from the at least one IAV vRNA segment. In some embodiments, the MSE is within an untranslated region (UTR) (e.g., 5′ UTR or 3′ UTR) of the IAV mRNA. In some embodiments, the MSE is within the open reading frame (ORF) of the IAV mRNA. In some embodiments, the first cellular environment is a first species, a first tissue, a first cell, or a first organelle, and the second cellular environment is a second species, a second tissue, a second cell, or a second organelle. In some embodiments, one or more IAV vRNA segments of the at least 7 IAV vRNA segments encodes a biological cargo. In some embodiments, the biological cargo is an antigen, a biologically active polypeptide (e.g., cytokine), a non-coding RNA (ncRNA), a marker polypeptide (e.g., green fluorescent protein (GFP)), one or more components of a gene editing system, or a Cre recombinase. In some embodiments, the target polypeptide is HA, NA, or a combination thereof.

In some embodiments, provided herein is an AIR vector, wherein the AIR vector comprises at least 7 IAV vRNA segments collectively encoding polypeptides, or functional mutants or derivatives thereof, of the IAV genome essential for AOR vector cell entry, cell exit and replication, wherein at least one IAV vRNA segment of the at least 7 IAV vRNA segments encodes an inhibitory domain configured such that a target polypeptide of the polypeptides of the IAV genome is not expressed or is inhibited in a first cellular environment, and wherein the target polypeptide is expressed and substantially functional in a second cellular environment. In some embodiments, the inhibitory domain is fused to the target polypeptide, wherein the inhibitory domain comprises a protease motif cleavable by a protease, wherein the protease is not expressed or expressed at a substantially lower level in the first cellular environment, and wherein the protease is expressed in the second cellular environment. In some embodiments, the inhibitory domain comprises a polypeptide that inhibits expression or function of the target polypeptide of the IAV genome. In some embodiments, the inhibitory domain is encoded within the ORF of an IAV mRNA produced from the at least one vRNA segment, wherein the IAV mRNA encodes the target polypeptide. In some embodiments, cleavage of the protease motif by a protease in the second cellular environment releases the inhibitory domain from the target polypeptide. In some embodiments, the target polypeptide is inhibited in the first cellular environment. In some embodiments, the first cellular environment is a first species and the second cellular environment is a second species. In some embodiments, one or more IAV vRNA segments of the at least 7 IAV vRNA segments encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In some embodiments, provided herein is an AIR vector, wherein the AIR vector comprises at least 7 IAV vRNA segments collectively encoding polypeptides, or functional mutants or derivatives thereof, of the IAV genome essential for AOR vector cell entry, cell exit and replication, wherein at least one IAV vRNA segment of the at least 7 IAV vRNA segments encodes a splicing motif configured such that a target polypeptide of the polypeptides of the IAV genome is not expressed or is not functional in a first cellular environment, and wherein the target polypeptide is expressed and substantially functional in a second cellular environment. In some embodiments, the splicing motif comprises a 5′-splice site and a 3′-splice site on an IAV mRNA produced from the least one IAV vRNA segment, wherein the IAV mRNA encodes the target polypeptide, wherein a region between the 5′-splice site and a 3′-splice site is not spliced out of the splicing motif in the first cellular environment such that no functional polypeptide is expressed, and wherein the region between the 5′-splice site and a 3′-splice site is spliced out of the splicing motif in the second cellular environment such that a functional polypeptide is expressed. In some embodiments, the splicing motif is within the open reading frame (ORF) of the IAV mRNA. In some embodiments, one or more IAV vRNA segments of the at least 7 IAV vRNA segments encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In some embodiments, provided herein is an AIR vector, wherein the AIR vector comprises at least 7 IAV vRNA segments collectively encoding polypeptides of the IAV genome, wherein at least one IAV vRNA segment of the at least 7 IAV vRNA segments comprises an IRES motif configured such that a target polypeptide of the polypeptides of the IAV genome is not expressed in a first cellular environment, and wherein the target polypeptide is expressed and substantially functional in a second cellular environment. In some embodiments, the IRES motif is on an IAV mRNA produced from the least one IAV vRNA segment, wherein the IAV mRNA encodes the target polypeptide, and wherein the IRES motif recruits a ribosome in the second cellular environment. In some embodiments, the first cellular environment is a first species and the second cellular environment is a second species. In some embodiments, the first species and the second species are different species. In some embodiments, the first species is human. In some embodiments, the IRES motif is immediately 5′ to a translational start site of the ORF encoding the target polypeptide on the IAV mRNA. In some embodiments, one or more IAV vRNA segments of the at least 7 IAV vRNA segments encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In some embodiments, provided herein is an AIR vector, wherein the AIR vector comprises at least 7 IAV vRNA segments collectively encoding polypeptides of the IAV genome (e.g., PB1, PB2, PA, HA, NP, NA, M1, M2, NS1, and NS2/NEP), wherein at least one IAV vRNA segment of the at least 7 IAV vRNA segments encodes a modulation moiety configured such that HA is not expressed or is inhibited in a first cellular environment, and wherein HA is expressed and substantially functional in a second cellular environment. In some embodiments, the modulation moiety is a miRNA Silencing Element (MSE), an inhibitory domain, a splicing motif, or an IRES motif. In some embodiments, the first cellular environment is a first species, a first tissue, a first cell, or a first organelle, and the second cellular environment is a second species, a second tissue, a second cell, or a second organelle. In some embodiments, one or more IA V vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase.

In some embodiments, provided herein is a composition comprising an AIR vector, wherein the AIR vector comprises at least 7 IAV vRNA segments collectively encoding polypeptides of the IAV genome (e.g., PB1, PB2, PA, HA, NP, NA, M1, M2, NS1, and NS2/NEP), wherein at least one IAV vRNA segment of the at least 7 IAV vRNA segments encodes a modulation moiety configured such that NA is not expressed or is inhibited in a first cellular environment, and wherein NA is expressed and substantially functional in a second cellular environment. In some embodiments, the modulation moiety is a miRNA Silencing Element (MSE), an inhibitory domain, a splicing motif, or an IRES motif. In some embodiments, the first cellular environment is a first species, a first tissue, a first cell, or a first organelle, and the second cellular environment is a second species, a second tissue, a second cell, or a second organelle. In some embodiments, one or more IAV vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase.

In other aspects, provided herein is a pharmaceutical composition comprising a composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes a modulation moiety configured such that a target polypeptide of the polypeptides of the IAV genome is not expressed or is inhibited in a first cellular environment, and wherein the target polypeptide is expressed and substantially functional in a second cellular environment. In some embodiments, the modulation moiety is a miRNA Silencing Element (MSE), an inhibitory domain, a splicing motif, or an IRES motif. In some embodiments, one or more IAV vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In further aspects, provided herein are methods of use of a composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes a modulation moiety configured such that a target polypeptide of the polypeptides of the IAV genome is not expressed or is inhibited in a first cellular environment, and wherein the target polypeptide is expressed and substantially functional in a second cellular environment. In some embodiments, the method is a method of treatment or prevention of a disease. In some embodiments, the method is a method of delivering a gene to an individual or a cell. In some embodiments, the method is a method of administering the AIR vector to a second cellular environment. In some embodiments, the modulation moiety is a miRNA Silencing Element (MSE), an inhibitory domain, a splicing motif, or an IRES motif. In some embodiments, one or more IAV vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

### A. Definitions

As used herein, the term “viral RNA (vRNA)” refers to genomic vRNA segments of the Orthomyxoviridae family. The term “influenza A virus (IAV) vRNA” refers to genomic IAV vRNA segments.

As used herein, the term “encoding” refers to encoding of an RNA (e.g., mRNA or non-coding RNA) or a polypeptide, and includes encoding in one or more parts (e.g., encoding with an interruption). The term “modulation moiety” can refer to either an RNA motif on an IAV mRNA or a polypeptide motif on a polypeptide encoded by the IAV mRNA.

The terms “miRNA motif”, “microRNA (miRNA) Silencing Element” or “MSE” are used herein interchangeably and refer to a nucleotide sequence within an mRNA that can bind to a specific miRNA and result in a measurable amount of post-transcriptional silencing of such mRNA (determined, e.g., by a decrease in mRNA and/or protein content). For post-transcriptional silencing to occur, MSE-miRNA sequence complementarity in most cases will include the seed sequence of the miRNA, which is typically comprised of nucleotides 1-7 or 2-8 of the miRNA, and additional complementarity following the seed sequence. In certain embodiments, perfect complementarity is desired.

The term “cellular environment” as used in the present disclosure refers to a species, a tissue, a cell, or an organelle. A “first cellular environment” and a “second cellular environment” are each either a first species and a second species, a first tissue and a second tissue, a first cell and a second cell, or a first organelle and a second organelle, respectively. A cellular environment may therefore include a cell, or an organelle within a cell, but does not necessarily refer to a cell.

The terms “microRNA” or “miRNA” as used herein refer to a structured RNA that is processed by the RNAse III components of the host cell. The resulting product is a ˜19-25 base pair endogenous single stranded RNA that regulates the expression of target mRNAs via a 7 base pair “seed” sequence (i.e., a miRNA target sequence at 5′ positions 1-7 or 2-8 of miRNA) as well as additional base pairing along the miRNA beyond the seed sequence. In some embodiments, the miRNA target sequence is selected such that the mean free energy (MFE) of the miRNA target sequence interaction with its corresponding miRNA is less than ˜20 kcal/mol.

Complementarity of an mRNA sequence to the “seed” of a miRNA is normally found in the 3′ untranslated region (3′ UTR). Bartel, Cell 116 (2): 281 (2004). miRNA regulation moderately affects global protein production resulting in a “fine tuning” of the cellular transcriptome. Baek et al., Nature 455 (7209): 64 (2008) and Selbach et al., Nature 455 (7209): 58 (2008).

The term “transfection” as used herein refers to the introduction or delivery of materials, such as purified nucleic acids (e.g., DNA or RNA), into cells. The transfection is often performed through methods that do not involve viral infection. The RNA-based systems provided herein can be transfected into cells, for example, by lipid nanoparticles or non-lipid based delivery vehicles.

The term “complementarity” means that a nucleic acid can form hydrogen bond(s) with another nucleic acid sequence by either traditional Watson-Crick or other non-traditional types of interactions such as Wobble-base pairing which permits binding of guanine and uracil. A percent complementarity indicates the percentage of residues in a nucleic acid molecule that can form hydrogen bonds with a second nucleic acid sequence.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, “a pharmaceutically acceptable carrier” refers to a pharmaceutically acceptable substrate, composition, or vehicle used in the process of drug delivery, which may have one or more ingredients including, but not limited to, excipient(s), binder(s), diluent(s), solvent(s), filler(s), and/or stabilizer(s).

An “individual” or “subject” or “animal”, as used herein, refers to vertebrates that support a negative strand RNA virus infection, specifically influenza virus infection, including, but not limited to, birds (such as water fowl and chickens) and members of the mammalian species, such as canine, feline, lupine, mustela, rodent (racine, murine, etc.), equine, bovine, ovine, caprine, porcine species, and primates, the latter including humans. In one embodiment, the subject is a human. In other embodiments, the subject is an insect. In one embodiment, the subject is a veterinary animal.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter.

As used herein and in the appended claims, the singular forms “a,” “an,” and “the” include plural referents unless the context clearly dictates otherwise.

B. AOR and AIR vectors

In one aspect, provided herein is a recombinant attenuated orthomyxovirus-based replicon (AOR) vector.

In one aspect, provided herein is an attenuated influenza A virus (IAV)-based replicon (AIR) vector.

The provided AIR vector allows normal IAV gene expression when replication occurs in cellular environments where conditions permit the expression and functional production of all necessary IAV proteins essential for viral vector cell entry, cell exit and replication, thus allowing scalable, clinical grade AIR vector production. Specifically, the AIR vector is incapable of generating HA or NA protein in a mammalian cell, effectively rendering it replication-incompetent in all cellular environments other than a selected cellular environment. As such, the AIR vector is only capable of a single-entry event mediated by the surface HA proteins that are derived from, for instance, in ovo replication. The AIR vector is additionally unable to spread from the initially infected cell, owing to the requirement of newly generated NA protein for viral egress from the cell and HA protein for subsequent entry into another susceptible cell. This safety feature reduces the potential risk of reversion due to selective pressure during viral replication and limits the actions of the vector to the initially targeted cells.

Because the AIR vector is unable to produce HA or NA protein in environments other than the selected cellular environment, there is no adverse antibody immune response directed to these proteins in other cellular environments. Therefore, the AIR vector may evade the host adaptive immune response and extend expression of a desired biological cargo. For example, the AIR vector can be engineered to carry a biological cargo (e.g., a foreign gene) inserted within the coding region of IAV mRNA produced from at least one IAV vRNA segment of the IAV genome, where the IAV mRNA encodes a polypeptide of the IAV genome. Alternatively, the biological cargo may be generated via use of a 2A ribosomal skipping peptide (e.g., P2A, T2A, E2A, F2A) to produce a polycistronic IAV mRNA. This advance enables the AIR vector to act as a gene delivery system for a variety of purposes, including expression of an antigen for induction of immunity against a pathogen, provision of a transgene to replace a defective function, or provision of a transgene to introduce an additional function to treat or prevent disease. The AIR vector may be further manipulated by targeting on IA V transcripts to achieve a desired expression profile in a selected cellular environment.

In some embodiments, the AIR vector comprises at least 7 IAV viral RNA (vRNA) segments collectively encoding polypeptides, or functional mutants or derivatives thereof, of the IAV genome essential for IAV vector cell entry, cell exit and replication, wherein at least one IAV vRNA segment of the at least 7 IAV vRNA segments encodes a modulation moiety configured such that a target polypeptide of the polypeptides of the IAV genome is not expressed or is inhibited in a first cellular environment, and wherein the target polypeptide is expressed and substantially functional in a second cellular environment. In some embodiments, a second target polypeptide of the polypeptides encoded by the IA V genome is not expressed or is inhibited in the first cellular environment, and wherein the second target polypeptide is expressed and substantially functional in the second cellular environment.

In some embodiments, the AIR vector exhibits reduced inflammation in the first cellular environment compared to a corresponding influenza A virus (IAV)-based vector without the modulation moiety. In some embodiments, the inflammation caused by the the AIR vector is reduced by greater than about 80% in the first cellular environment compared to a corresponding IAV-based vector without the modulation moiety, such as reduced by greater than any of about 85%, 90%, 91%, 92%, 93%, 94%, 95%, 96%, 97%, 98%, 99%, or more. In some embodiments, the inflammation caused by the AIR vector is reduced by less than about 99% in the first cellular environment compared to a corresponding IAV-based vector without the modulation moiety, such as reduced by less than any of about 98%, 97%, 96%, 95%, 94%, 93%, 92%, 91%, 90%, 85%, 80%, or less. In some embodiments, the inflammation induced by the the AIR vector is reduced by any of about 80%, 85%, 90%, 91%, 92%, 93%, 94%, 95%, 96%, 97%, 98%, or 99% in the first cellular environment compared a corresponding IAV-based vector without the modulation moiety. Inflammation can be measured by any technique known in the art. For instance, in some embodiments, inflammation is measured by IgG antibody response to a targeted IAV protein (e.g., the target polypeptide of the polypeptides of the IAV genome, e.g., HA and/or NA). In some embodiments, inflammation is measured by IgM antibody response to a targeted IAV protein (e.g., the target polypeptide of the polypeptides of the IAV genome, e.g., HA and/or NA). In some embodiments, inflammation is measured by monitoring of the transcriptional host response.

In some embodiments, the AIR vector has decreased replication in the first cellular environment compared to replication of a corresponding IAV-based vector without the modulation moiety. In some embodiments, replication of the AIR vector in the first cellular environment is decreased any of about 1-, 2-, 3-, 4-, 5-, 10-fold, or more, compared to replication of a corresponding IAV-based vector without the modulation moiety. In some embodiments, the AIR vector is not capable of replicating in the first cellular environment. In some embodiments, the AIR vector is capable of replicating in the second cellular environment.

In some embodiments, the target polypeptide (e.g., HA and/or NA) is not expressed or is inhibited in the first cellular environment for between about 2 to about 14 days, such as for between about 2 to about 8 days, between about 5 to about 10 days, or between about 8 to about 14 days. In some embodiments, the target polypeptide is not expressed or is inhibited in the first cellular environment for greater than about 2 days, such as greater than any of about 3, 4, 5, 6, 7, 8, 9, 10, 11, 12, 13, 14, or more, days. In some embodiments, the target polypeptide is not expressed or is inhibited in the first cellular environment for less than about 14 days, such as less than any of about 13, 12, 11, 10, 9, 8, 7, 6, 5, 4, 3, 2, or fewer days.

i. IAV

The present invention provides an attenuated IAV-based vector (e.g., AIR vector) encoding polypeptides of the IAV genome. In some embodiments, the IAV may be any IAV known in the art, variant, or reassortant thereof.

IAV is a member of the Orthomyxoviridae family of viruses. Orthomyxoviridae are a group of related, yet antigenically and genetically diverse viruses. In mammals, such as humans, Orthomyxoviridae cause respiratory tract infections that can range from mild to lethal. Orthomyxoviridae viruses encode their genome in segments of negative-sense RNA. In some embodiments, Orthomyxoviridae contain six to eight segments of linear negative-sense single stranded RNA. They have a total genome length that is between about 10,000 and about 14,600 nucleotides. In some embodiments, a virus of the Orthomyxoviridae family is an influenza virus (e.g., an Alphainfluenzavirus, Betainfluenzavirus, Gammainfluenzavirus, or a Deltainfluenzavirus). There are four type of influenza viruses: Type A (IAV), Type B (IBV), Type C (ICV), and Type D (IDV). Of the four types of influenza virus, three types (A, B, and C) affect humans. In some embodiments, the virus is IAV, IBV, ICV, IDV, or a variant, subtype, or reassortant thereof.

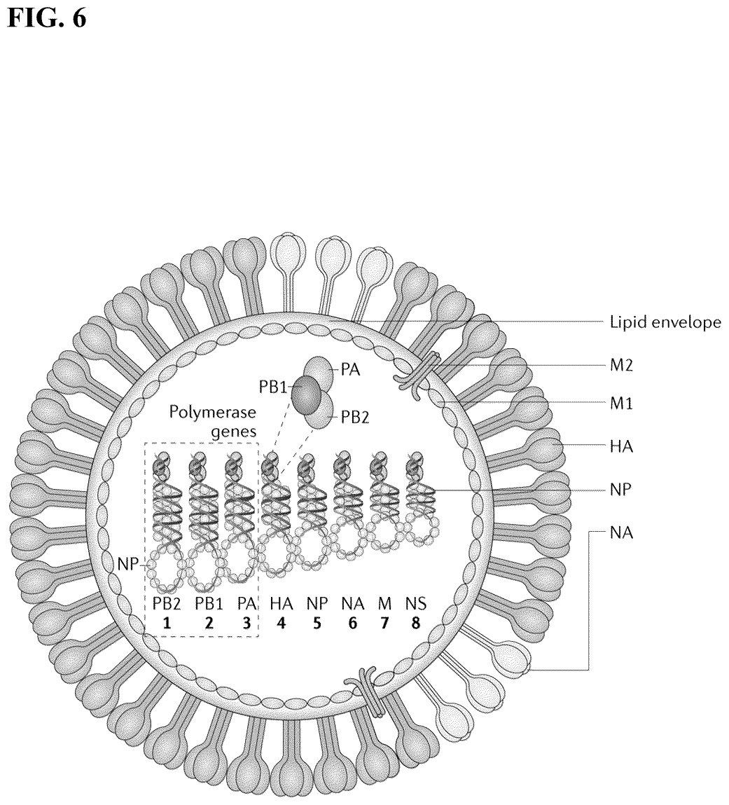

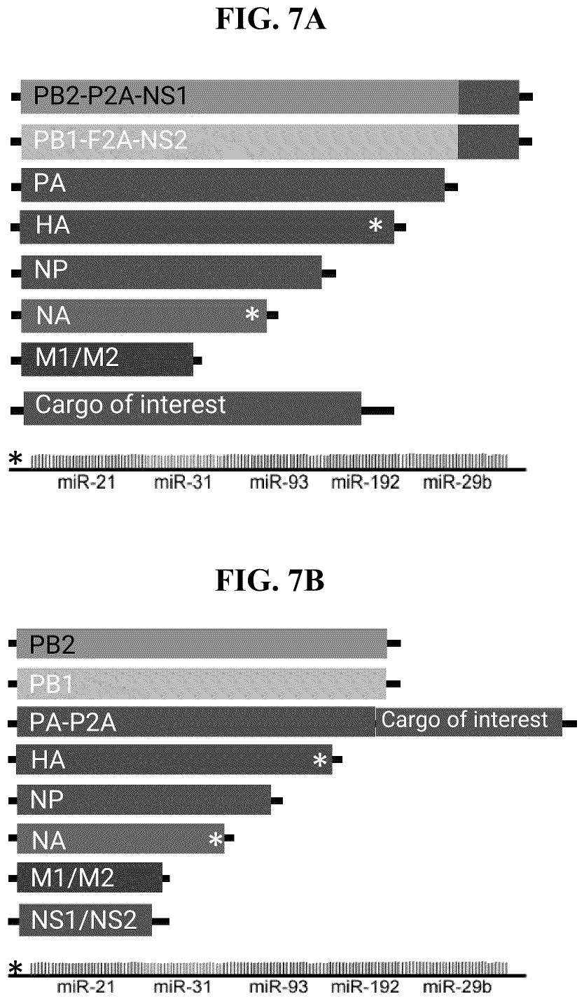

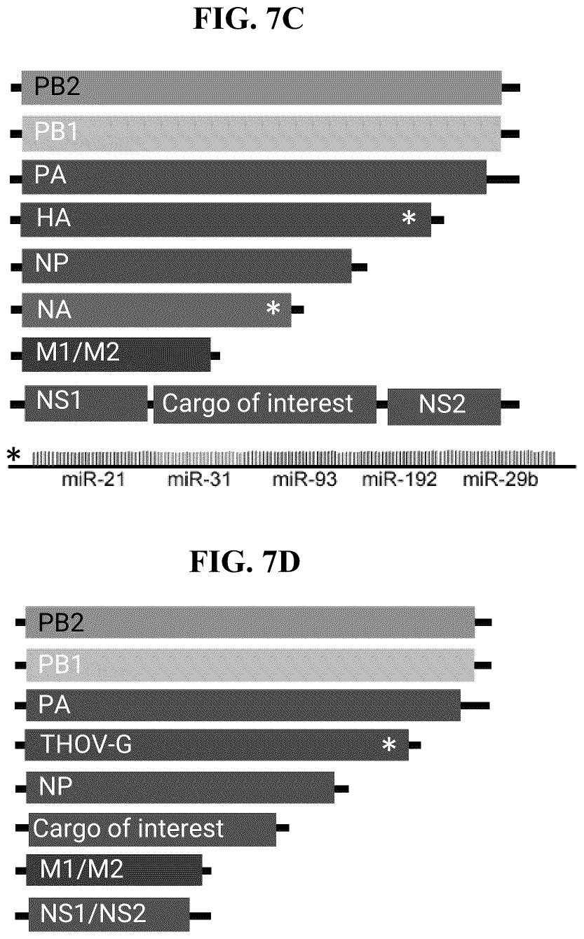

As shown in the left panel of FIG. 6, IAV is comprised of a segmented genome comprising 8 negative-sense IAV viral RNA (vRNA) segments, making up the entire viral genome of IAV. The negative-sense IAV vRNAs are the complement sequence to their open reading frames (ORFs). Segments 1-6 generate one major protein each, whereas segment 7-8 undergo splicing to make two proteins per IAV vRNA segment. Generation of IAV mRNA demands the viral RNA-dependent RNA polymerase (RdRp), which recognizes the terminal ends of each vRNA, and comprises three subunits (PA, PB1 and PB2). The segments of IA V illustrated in FIG. 6 are as follows: segment 1-PB2 (core RdRp component); segment 2-PB1 (core RdRp component); segment 3-PA (core RdRp component); segment 4-HA (viral attachment protein); segment 5-nucleoprotein (“NP,” RNA binding protein for viral segments); segment 6-NA (viral release protein); segment 7-matrix protein 1 (“M1,” virion structural protein) and matrix protein 2 (“M2,” ion channel required for entry and egress); and, segment 8-non-structural protein 1 (“NS1,” antagonist of host antiviral defenses) and non-structural protein 2 (“NS2,” RdRp modulation and virus egress). Viruses of different genera of the Orthomyxoviridae family may have different ORFs encoding their viral components, and different vRNA segments in their genome comprising such ORFs (i.e., less than or more than 8 vRNA segments). In some embodiments, the polypeptides encoded by the IA V genome are PB1, PB2, PA, HA, NP, NA, M1, M2, NS1, and NS2/NEP.

In some embodiments, non-essential genes can be omitted, such as NS1 which is an antagonist of the cell's antiviral defense system. In some embodiments, the polypeptides encoded by the IAV genome are PB1, PB2, PA, HA, NP, NA, M1, M2, and NS2/NEP.

In some embodiments, the AIR vector comprises IAV vRNA segments collectively encoding polypeptides of the IAV genome. Each IAV vRNA segment of the at least 7 IAV vRNA segments share a common organization: a central ORF (in the antisense orientation), which is flanked at the 3′ and 5′ ends by untranslated regions (UTRs). The UTRs of IAV vRNA segments comprise highly conserved motif sequences between the IAV vRNAs at the 3′ and 5′ termini of every IAV vRNA segment, and segment specific non-coding regions (ssNCRs), the length and sequences of which are specific to each IAV vRNA segment. In some embodiments, specific sequences within the non-coding regions and the adjacent coding regions at the 3′ and 5′ ends of each vRNA segment comprise one or more packaging sequences for the packaging of the vRNA segment into a virion (e.g., packaging signal). In some embodiments, specific sequences within the internal coding regions comprise one or more packaging sequences for the packaging of the vRNA segment into a virion. In some embodiments, the AIR vector comprises the packing sequences from each of the vRNA segments collectively encoding polypeptides of the IAV genome (e.g., PB1, PB2, PA, HA, NP, NA, M1, M2, NS1, and NS2/NEP).

In some embodiments, the AIR vector comprises at least 7 IAV vRNA segments collectively encoding polypeptides of the IAV genome. In some embodiments, the AIR vector comprises 8 IAV vRNA segments collectively encoding polypeptides of the IAV genome. In some embodiments, the AIR vector comprises more than 8 IAV vRNA segments collectively encoding polypeptides of the IAV genome. In some embodiments, each IAV vRNA segment of the at least 7 IAV vRNA segments encodes one polypeptide of the IAV genome. In some embodiments, each IAV vRNA segment of the at least 7 IAV vRNA segments may encode one or more polypeptides of the IAV genome. In some embodiments, at least two of the IAV vRNA segments each encode two polypeptides of the IAV genome.

In some embodiments, the AIR vector comprises 8 IAV vRNA segments. In some embodiments, IAV vRNA segment 1 is about 2.3 kB in length. In some embodiments, IAV vRNA segment 2 is about 2.3 KB in length. In some embodiments, IAV vRNA segment 3 is about 2.1 kB in length. In some embodiments, IAV vRNA segment 4 is about 1.5 kB in length. In some embodiments, IAV vRNA segment 5 is about 1.4 KB in length. In some embodiments, IAV vRNA segment 6 is about 1.0 KB in length. In some embodiments, IAV vRNA segment 7 is about 0.9 kB in length. In some embodiments, IAV vRNA segment 8 is about 0.8 KB in length. It should be understood that the IAV vRNA segments may be shorter or longer than the above exemplary embodiments based on AIR construction. For examples, in some embodiments where viral ORFs are moved to different IAV vRNA segments via splicing or use of a ribosomal skipping site (e.g., P2A), the IAV vRNA segments may be longer whereas the IAV vRNA segments originally encoding those ORFs (which may be repurposed for carrying a biological cargo) may be longer or shorter compared to the unmodified IAV vRNA segment.

In some embodiments, the 8 vRNA segments collectively encode PB1, PB2, PA, HA, NP, NA, M1, M2, NS1, and NS2/NEP. In some embodiments, the AIR vector comprises IAV vRNA segment 1 for PB2, IAV vRNA segment 2 for PB1, IAV vRNA segment 3 for PA, IAV vRNA segment 4 for HA, IAV vRNA segment 5 for NP, IAV vRNA segment 6 for NA, IAV vRNA segment 7 for M1 and M2, and IAV vRNA segment 8 for NS1 and NS2. In some embodiments, the IAV vRNA segments collectively encode the IAV genome.

In some embodiments, the IAV vector may be a pseudotype influenza A virus.

In some embodiments, IAV HA and/or NA, or functional mutants or derivatives thereof, may be optionally replaced by one or more heterologous envelope glycoproteins that can mediate IAV cell entry and egress. In some embodiments, the heterologous envelope glycoprotein is a vesiculovirus G protein, thogotovirus G protein, or a functional mutant or derivative thereof. In some embodiments, IAV HA and/or NA, or functional mutants or derivatives thereof, are replaced with a single glycoprotein (e.g., a vesiculovirus G protein, thogotovirus G protein) so that the unused vRNA gene segment (either HA or NA) can be used to encode a biological cargo. For example, the single glycoprotein (e.g., a vesiculovirus G protein, thogotovirus G protein) may be encoded by IAV vRNA segment 4 (HA) or 6 (NA) wherein the other segment can be used to encode a biological cargo.

ii. Modulation Moiety

At least one IAV vRNA segment of the at least 7 vRNA segments encodes a modulation moiety. The modulation moiety is configured such that a target polypeptide (e.g., a first target polypeptide) of the polypeptides of the IAV genome is not expressed or is inhibited in a first cellular environment, but the target polypeptide is expressed and substantially functional in a second cellular environment. The modulation moiety encompasses various mechanisms contemplated herein. In some embodiments, at least one IAV vRNA segment of the at least 7 vRNA segments encodes a modulation moiety. In some embodiments, two, three, four, or more, IAV vRNA segment of the at least 7 vRNA segments encodes a modulation moiety. In some embodiments, one IAV vRNA segment of the at least 7 vRNA segments encodes a modulation moiety. In some embodiments, two IAV vRNA segments of the at least 7 vRNA segments encodes a modulation moiety. In some embodiments, a first IAV vRNA segment of the at least 7 vRNA segments encodes a first modulation moiety, and a second IAV vRNA segment of the at least 7 vRNA segments encodes a second modulation moiety. In some embodiments, the first modulation moiety and the second modulation moiety are the same modulation moiety. In some embodiments, the first modulation moiety and the second modulation moiety are different modulation moieties.

In some embodiments, the modulation moiety impacts one target polypeptide of the polypeptides of the IAV genome (e.g., the target polypeptide that is not expressed or is inhibited in the first cellular environment but is expressed and substantially functional in the second cellular environment). In some embodiments, the modulation moiety impacts more than one polypeptide (e.g., there is more than one target polypeptide). In some embodiments, the target polypeptide of the polypeptides of the IAV genome is a first polypeptide. In some embodiments, the first target polypeptide of the polypeptides encoded by the IAV genome is not expressed or is inhibited in the first cellular environment. In some embodiments, the first target polypeptide is expressed and substantially functional in the second cellular environment. In some embodiments, the first target polypeptide of the polypeptides of the IAV genome is HA. In some embodiments, the first target polypeptide of the polypeptides of the IAV genome is NA.

In some embodiments, additional target polypeptides of the polypeptides of the IAV genome are not expressed or are inhibited in the first cellular environment but are expressed and substantially functional in the second cellular environment. For example, in some embodiments, a second target polypeptide of the polypeptides encoded by the IAV genome is not expressed or is inhibited in the first cellular environment. In some embodiments, the second target polypeptide is expressed and substantially functional in the second cellular environment. In some embodiments, the first target polypeptide of the polypeptides of the IAV genome is HA, and the second target polypeptide of the polypeptides of the IAV genome is NA. In some embodiments, the first target polypeptide of the polypeptides of the IAV genome is NA, and the second target polypeptide of the polypeptides of the IAV genome is HA.

In some embodiments, the modulation moiety is an RNA segment on an IAV mRNA. In some embodiments, the modulation moiety is on an IAV mRNA produced from the least one IAV vRNA segment. In some embodiments, the IAV mRNA encodes the target polypeptide that is not expressed or is inhibited in the first cellular environment but is expressed and substantially functional in the second cellular environment (e.g., a first polypeptide). In some embodiments, the modulation moiety is 3′ to a stop codon of the IAV mRNA. In some embodiments, the modulation moiety is within the open reading frame (ORF) of the IAV mRNA.

In some embodiments, the modulation moiety is an amino acid sequence on the target polypeptide encoded by the IAV mRNA. In some embodiments, the modulation moiety is on the target polypeptide encoded by the IAV mRNA. In some embodiments, the IAV mRNA is produced from the at least one IAV vRNA segment.

In some embodiments, a cellular environment is a species. In some embodiments, the species is mammal, chicken, monkey, canine, insect, plant, yeast, or bacteria. In some embodiments, the species is human. In some embodiments, the first cellular environment is a first species and the second cellular environment is a second species. In some embodiments, the first species and the second species are different species. In some embodiments, the first species is mammal. In some embodiments, the first species is human. In some embodiments, the second species is chicken, monkey, canine, insect, plant, yeast, or bacteria. In some embodiments, the first species is human and the second species is chicken.

In some embodiments, a cellular environment is a tissue. In some embodiments, the tissue is connective tissue, epithelial tissue, muscle tissue, or nervous tissue. In some embodiments, the first cellular environment is a first tissue and the second cellular environment is a second tissue. In some embodiments, the first tissue and the second tissue are from the same species. In some embodiments, the first tissue and the second tissue are from different species. In some embodiments, the second tissue is from a human.

In some embodiments, a cellular environment is a cell. In some embodiments, the cell is an animal cell, a plant cell, an insect cell (e.g., a mosquito cell), a bacteria cell, or a yeast cell. In some embodiments, the cell is from Nicotiana benthamiana, Arabidopsis thaliana, Medicago sativa, Zea mays, or Solanum tuberosum. In some embodiments, the cell is a fibroblast, kidney cell, embryonic cell, retinal cell, or an ovary cell. In some embodiments, the cell is a chicken fibroblast DF1, Madin-Darby Canine Kidney (MCK) cell, an African green monkey kidney cell (Vero), a human PER-C6 cell, or a C6/36 mosquito cell. In some embodiments, the first cellular environment is a first cell and the second cellular environment is a second cell. In some embodiments, the first cell and the second cell are from the same species. In some embodiments, the first cell and the second cell are from different species. In some embodiments, the first cell and the second cell are from the same tissue. In some embodiments, the first cell and the second cell are from different tissues. In some embodiments, the first cell is a human cell and the second cell is an embryonated chicken egg.

In some embodiments, a cellular environment is an organelle. In some embodiments, the organelle is a mitochondria, nucleus, endoplasmic reticulum, ribosome, centriole, lysosome, or the Golgi body. In some embodiments, the first cellular environment is a first organelle and the second cellular environment is a second organelle. In some embodiments, the first organelle and the second organelle are from the same species. In some embodiments, the first organelle and the second organelle are from different species. In some embodiments, the first organelle and the second organelle are from the same cell. In some embodiments, the first organelle and the second organelle are from different cells.

Exemplary modulation moieties are described below.

a. miRNA Silencing Element (MSE)

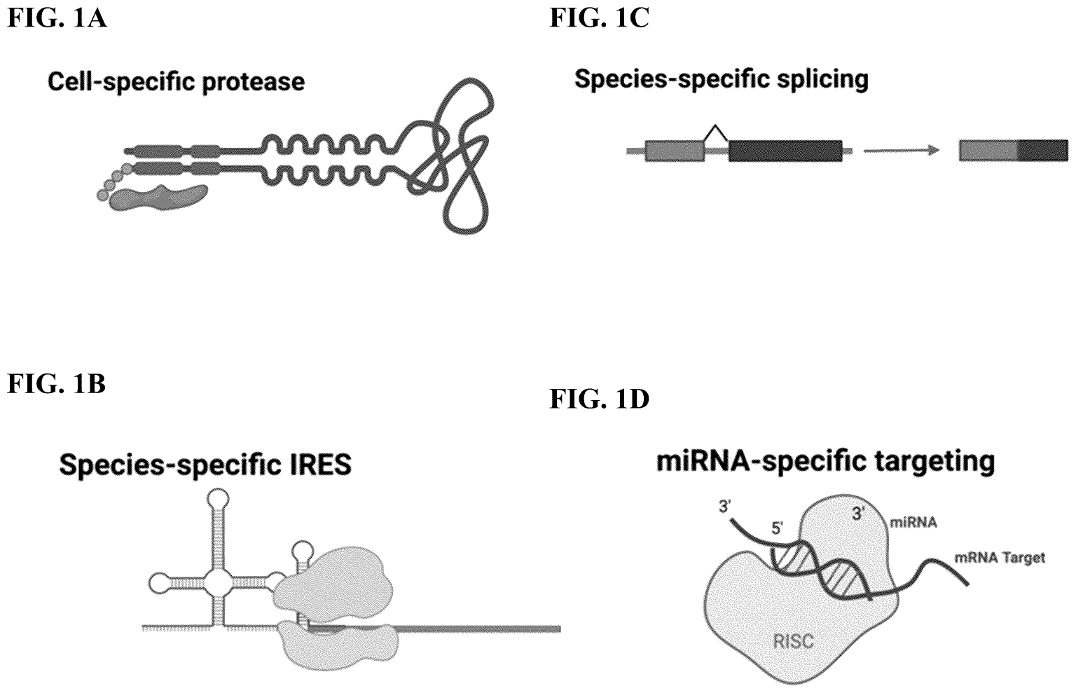

In some aspects, a modulation moiety of the AIR vector is a miRNA Silencing Element (MSE). This embodiment of the AIR vector exploits cell—and/or species-specific miRNA expression. As shown in FIG. 1D, unique expression of a given miRNA, or group of miRNAs, may be leveraged to selectively silence a desired transcription in an inverse manner from where expression occurs.

Thus, in some aspects, provided herein is an AIR vector, wherein the AIR vector comprises at least 7 IAV vRNA segments collectively encoding polypeptides of the IAV genome, wherein at least one IAV vRNA segment of the at least 7 IAV vRNA segments encodes a MSE configured such that a target polypeptide of the polypeptides of the IAV genome is not expressed or is inhibited in a first cellular environment, and wherein the target polypeptide is expressed and substantially functional in a second cellular environment. In some embodiments, the MSE is on an IAV mRNA produced from the least one IAV vRNA segment, wherein the IAV mRNA encodes the target polypeptide, and wherein the MSE is targeted by a miRNA in the first cellular environment. In some embodiments, the target polypeptide is HA, NA, or a combination thereof.

In some embodiments, the miRNA is encoded on a IAV vRNA segment of the at least 7 IAV vRNA segments. In some embodiments, the miRNA is encoded on the same IAV vRNA segment on which the MSE is encoded. In some embodiments, the miRNA is encoded on a different IAV vRNA segment that the IAV vRNA segment on which the MSE is encoded.

In some embodiments, the miRNA is not encoded on an IAV vRNA segment of the at least 7 IAV vRNA segments. In some embodiments, the miRNA is provided to the first cellular environment. In some embodiments, the miRNA is provided to the first cellular environment and the second cellular environment. In some embodiments, the miRNA is endogenous to the first cellular environment.

In some embodiments, the miRNA decreases expression of the target polypeptide in the first cellular environment. In some embodiments, the miRNA decreases expression of the target polypeptide in the first cellular environment any of about 1-, 2-, 3-, 4-, 5-, 10-fold, or greater, compared to expression of the target polypeptide in a cellular environment that does not contain the miRNA. In some embodiments, the miRNA silences expression of the target polypeptide in the first cellular environment. In some embodiments, expression of the miRNA in the first cellular environment is at least about 2 times higher than the expression of the miRNA in the second cellular environment, such as any of about 3, 4, 5, 6, 7, 8, 9, 10, or more, times higher than the expression of the miRNA in the second cellular environment. In some embodiments, the miRNA is not expressed in the second cellular environment. In some embodiments, the first cellular environment is a first species, a first tissue, a first cell, or a first organelle, and the second cellular environment is a second species, a second tissue, a second cell, or a second organelle.

In some embodiments, the MSE is on the IAV mRNA produced from the at least one IAV vRNA segment. In some embodiments, the MSE is within an untranslated region (UTR) (e.g., 5′ UTR or 3′ UTR) of the IAV mRNA. In some embodiments, the MSE is 3′ to the stop codon of the IAV mRNA. In some embodiments, the MSE is within the open reading frame (ORF) of the IAV mRNA.

In some embodiments, the at least one IAV vRNA segment of the at least 7 IAV vRNA segments encodes two or more miRNA Silencing Elements (MSEs), such as 3, 4, 5, 6, 7, 8, 9, 10, or more. In some embodiments, the at least one IAV vRNA segment of the at least 7 IAV vRNA segments encodes two MSEs. In some embodiments, the at least one IAV vRNA segment of the at least 7 IAV vRNA segments encodes three MSEs. In some embodiments, the at least one IAV vRNA segment of the at least 7 IAV vRNA segments encodes four MSEs. In some embodiments, the at least one IAV vRNA segment of the at least 7 IAV vRNA segments encodes five MSEs. In some embodiments, the at least one IAV vRNA segment of the at least 7 IAV vRNA segments encodes six MSEs. In some embodiments, the at least one IAV vRNA segment of the at least 7 IAV vRNA segments encodes seven MSEs. In some embodiments, the at least one IAV vRNA segment of the at least 7 IAV vRNA segments encodes eight MSEs. In some embodiments, the at least one IAV vRNA segment of the at least 7 IAV vRNA segments encodes nine MSEs. In some embodiments, the at least one IAV vRNA segment of the at least 7 IAV vRNA segments encodes ten MSEs.

In some embodiments, the miRNA is from a vertebrate. In some embodiments, the miRNA is mir-21, mir-31, mir-192, mir-29a, mir-29b, mir-125a, mir-320a, mir-151, mir-1974, mir-331, mir-106b, mir-224, let-7e, mir-574, mir-320c-1, mir-215, mir-92b, mir-361, mir-378, mir-424, mir-423, mir-320b, mir-339, mir-1259, mir-99b, mir-28, mir-26b, mir-194-2, mir-183, mir-582, mir-1274a, mir-342, mir-1249, mir-1307, mir-494, mir-1180, mir-542, mir-452, mir-376c, mir-374b, mir-409, mir-194-1, mir-197, mir-34b, mir-152, let-7d, mir-345, mir-362, mir-505, mir-421, mir-487b, mir-625, mir-132, mir-503, mir-181c, mir-376a-1, mir-1280, mir-376a-2, mir-652, miR-124, miR-142, miR-1, miR-122, miR-200, miR-133a, miR-375, miR-143, miR-145, and miR-184, let-7a, miR-206, miR-155, miR-208, miR-499, miR-192, miR-7, miR-9, miR-223, miR-150, miR-137, miR-134, let-7a-3p, let-7a-5p, let-7b-3p, let-7b-5p, let-7c-3p, let-7c-5p, let-7d-3p, let-7d-5p, let-7e-3p, let-7e-5p, let-7f-1-3p, let-7f-2-3p, let-7f-5p, let-7g-3p, let-7g-5p, let-7i-3p, let-7i-5p, miR-1-3p, miR-1-5p, miR-100-3p, miR-100-5p, miR-101-2-5p, miR-101-3p, miR-101-5p, miR-10226, miR-10392-3p, miR-10392-5p, miR-10393-3p, miR-10393-5p, miR-10394-3p, miR-10394-5p, miR-10395-3p, miR-10395-5p, miR-10396a-3p, miR-10396a-5p, miR-10396b-3p, miR-10396b-5p, miR-10397-3p, miR-10397-5p, miR-10398-3p, miR-10398-5p, miR-10399-3p, miR-10399-5p, miR-103a-1-5p, miR-103a-2-5p, miR-103a-3p, miR-103b, miR-10400-3p, miR-10400-5p, miR-10401-3p, miR-10401-5p, miR-105-3p, miR-105-5p, miR-10522-5p, miR-10523-5p, miR-10524-5p, miR-10525-3p, miR-10526-3p, miR-10527-5p, miR-106a-3p, miR-106a-5p, miR-106b-3p, miR-106b-5p, miR-107, miR-10a-3p, miR-10a-5p, miR-10b-3p, miR-10b-5p, miR-11181-3p, miR-11181-5p, miR-11399, miR-11400, miR-11401, miR-1178-3p, miR-1178-5p, miR-1179, miR-1180-3p, miR-1180-5p, miR-1181, miR-1182, miR-1183, miR-1184, miR-1185-1-3p, miR-1185-2-3p, miR-1185-5p, miR-1193, miR-1197, miR-1199-3p, miR-1199-5p, miR-1200, miR-1202, miR-1203, miR-1204, miR-1205, miR-1206, miR-1207-3p, miR-1207-5p, miR-1208, miR-12113, miR-12114, miR-12115, miR-12116, miR-12117, miR-12118, miR-12119, miR-12120, miR-12121, miR-12122, miR-12123, miR-12124, miR-12125, miR-12126, miR-12127, miR-12128, miR-12129, miR-12130, miR-12131, miR-12132, miR-12133, miR-12135, miR-12136, miR-122-3p, miR-122-5p, miR-1224-3p, miR-1224-5p, miR-1225-3p, miR-1225-5p, miR-1226-3p, miR-1226-5p, miR-1227-3p, miR-1227-5p, miR-1228-3p, miR-1228-5p, miR-1229-3p, miR-1229-5p, miR-122b-3p, miR-122b-5p, miR-1231, miR-1233-3p, miR-1233-5p, miR-1234-3p, miR-1236-3p, miR-1236-5p, miR-1237-3p, miR-1237-5p, miR-1238-3p, miR-1238-5p, miR-124-3p, miR-124-5p, miR-1243, miR-1244, miR-1245a, miR-1245b-3p, miR-1245b-5p, miR-1246, miR-1247-3p, miR-1247-5p, miR-1248, miR-1249-3p, miR-1249-5p, miR-1250-3p, miR-1250-5p, miR-1251-3p, miR-1251-5p, miR-1252-3p, miR-1252-5p, miR-1253, miR-1255a, miR-1255b-2-3p, miR-1255b-5p, miR-1256, miR-1257, miR-1258, miR-125a-3p, miR-125a-5p, miR-125b-1-3p, miR-125b-2-3p, miR-125b-5p, miR-126-3p, miR-126-5p, miR-1260a, miR-1260b, miR-1261, miR-1262, miR-1263, miR-1264, miR-1265, miR-1266-3p, miR-1266-5p, miR-1267, miR-1268a, miR-1268b, miR-1269a, miR-1269b, miR-127-3p, miR-127-5p, mmiR-1270, miR-1271-3p, miR-1271-5p, miR-1272, miR-1273c, miR-1273h-3p, miR-1273h-5p, miR-1275, miR-1276, miR-1277-3p, miR-1277-5p, miR-1278, miR-1279, miR-128-1-5p, miR-128-2-5p, miR-128-3p, miR-1281, miR-1282, miR-1283, miR-1284, miR-1285-3p, miR-1285-5p, miR-1286, miR-1287-3p, miR-1287-5p, miR-1288-3p, miR-1288-5p, miR-1289, miR-129-1-3p, miR-129-2-3p, miR-129-5p, miR-1290, miR-1291, miR-1292-3p, miR-1292-5p, miR-1293, miR-1294, miR-1295a, miR-1295b-3p, miR-1295b-5p, miR-1296-3p, miR-1296-5p, miR-1297, miR-1298-3p, miR-1298-5p, miR-1299, miR-1301-3p, miR-1301-5p, miR-1302, miR-1303, miR-1304-3p, miR-1304-5p, miR-1305, miR-1306-3p, miR-1306-5p, miR-1307-3p, miR-1307-5p, miR-130a-3p, miR-130a-5p, miR-130b-3p, miR-130b-5p, miR-132-3p, miR-132-5p, miR-1321, miR-1322, miR-1323, miR-1324, miR-133a-3p, miR-133a-5p, miR-133b, miR-134-3p, miR-134-5p, miR-1343-3p, miR-1343-5p, miR-135a-2-3p, miR-135a-3p, miR-135a-5p, miR-135b-3p, miR-135b-5p, miR-136-3p, miR-136-5p, miR-137-3p, miR-137-5p, miR-138-1-3p, miR-138-2-3p, miR-138-5p, miR-139-3p, miR-139-5p, miR-140-3p, miR-140-5p, miR-141-3p, miR-141-5p, miR-142-3p, miR-142-5p, or miR-143-3p. In some embodiments, the miRNA is mir-21, mir-31, mir-192, mir-29a, mir-29b, mir-125a, mir-320a, mir-151, mir-1974, mir-331, mir-106b, mir-224, let-7e, mir-574, mir-320c-1, mir-215, mir-92b, mir-361, mir-378, mir-424, mir-423, mir-320b, mir-339, mir-1259, mir-99b, mir-28, mir-26b, mir-194-2, mir-183, mir-582, mir-1274a, mir-342, mir-1249, mir-1307, mir-494, mir-1180, mir-542, mir-452, mir-376c, mir-374b, mir-409, mir-194-1, mir-197, mir-34b, mir-152, let-7d, mir-345, mir-362, mir-505, mir-421, mir-487b, mir-625, mir-132, mir-503, mir-181c, mir-376a-1, mir-1280, mir-376a-2, mir-652, miR-124, miR-142, miR-1, miR-122, miR-200, miR-133a, miR-375, miR-143, miR-145, and miR-184, let-7a, miR-206, miR-155, miR-208, miR-499, miR-192, miR-7, miR-9, miR-223, miR-150, miR-137, or miR-134, or any combination thereof.

In some embodiments, the miRNA is miR-21, miR-31, miR-192, miR-93, miR-29b, or any combination thereof.

In some embodiments, the miRNAs are miR-21 and miR-31. In some embodiments, the miRNAs are miR-21 and miR-192. In some embodiments, the miRNAs are miR-21 and miR-93. In some embodiments, the miRNAs are miR-21 and miR-29b. In some embodiments, the miRNAs are miR-31 and miR-192. In some embodiments, the miRNAs are miR-31 and miR-93. In some embodiments, the miRNAs are miR-31 and miR-29b. In some embodiments, the miRNAs are miR-192 and miR-93. In some embodiments, the miRNAs are miR-192 and miR-29b. In some embodiments, the miRNAs are miR-93 and miR-29b.

In some embodiments, the miRNAs are miR-21, miR-31, and miR-192. In some embodiments, the miRNAs are miR-21, miR-31, and miR-93. In some embodiments, the miRNAs are miR-21, miR-31, and miR-29b. In some embodiments, the miRNAs are miR-21, miR-192, and miR-93. In some embodiments, the miRNAs are miR-21, miR-192, and miR-29b. In some embodiments, the miRNAs are miR-21, miR-93, and miR-29b. In some embodiments, the miRNAs are miR-31, miR-192, and miR-93. In some embodiments, the miRNAs are miR-31, miR-93, and miR-29b. In some embodiments, the miRNAs are miR-31, miR-192, and miR-29b. In some embodiments, the miRNAs are miR-192, miR-93, and miR-29b.

In some embodiments, the miRNAs are miR-21, miR-31, miR-192, and miR-93. In some embodiments, the miRNAs are miR-21, miR-31, miR-192, and miR-29b. In some embodiments, the miRNAs are miR-21, miR-31, miR-29b, and miR-93. In some embodiments, the miRNAs are miR-21, miR-29b, miR-192, and miR-93. In some embodiments, the miRNAs are miR-29b, miR-31, miR-192, and miR-93.

In some embodiments, the miRNA is not present in the allantois of a fertilized egg.

b. Inhibitory Domain

In some aspects, a modulation moiety of the AIR vector is an inhibitory domain fused to a target polypeptide of the polypeptides of the IAV genome. This embodiment of the AIR vector leverages cell-specific proteases. As shown in FIG. 1A, the inhibitory domain further comprises a protease motif situated between the inhibitory domain and the target polypeptide of the IAV genome. The inhibitory domain prevents the function of the target polypeptide of the IAV genome. When a protease recognizes the protease motif, the protease motif is cleaved, thus releasing the inhibitory domain from the target polypeptide of the IAV genome. Once cleaved the target polypeptide of the IAV genome is substantially functional in that cellular environment, thereby allowing viral replication exclusively in cellular environments where the protease is present.

Thus, in some aspects, provided herein is an AIR vector, wherein the AIR vector comprises at least 7 IAV vRNA segments collectively encoding polypeptides of the IAV genome, wherein at least one IAV vRNA segment of the at least 7 IAV vRNA segments encodes an inhibitory domain configured such that a target polypeptide of the polypeptides of the IAV genome is not expressed or is inhibited in a first cellular environment, and wherein the target polypeptide is expressed and substantially functional in a second cellular environment. In some embodiments, the inhibitory domain fused to the polypeptide, wherein the inhibitory domain comprises a protease motif cleavable by a protease, and wherein the protease is not expressed in the first cellular environment. In some embodiments, the target polypeptide is HA, NA, or a combination thereof.

In some embodiments, the protease is encoded on a IAV vRNA segment of the at least 7 IAV vRNA segments. In some embodiments, the protease is encoded on the same IAV vRNA segment on which the inhibitory domain is encoded. In some embodiments, the protease is encoded on a different IAV vRNA segment that the IAV vRNA segment on which the inhibitory domain is encoded.

In some embodiments, the protease is not encoded on an IAV vRNA segment of the at least 7 IAV vRNA segments. In some embodiments, the protease is provided to the second cellular environment. In some embodiments, the protease is provided to the first cellular environment and the second cellular environment. In some embodiments, the protease is endogenous to the second cellular environment.

In some embodiments, the inhibitory domain comprises a polypeptide that inhibits expression or function of the target polypeptide of the IAV genome. In some embodiments, the inhibitory domain comprises a negatively charged polypeptide. In some embodiments, the inhibitory domain comprises a positively charged polypeptide. In some embodiments, the inhibitory domain is encoded within the ORF of an IAV mRNA produced from the at least one vRNA segment, wherein the IAV mRNA encodes the polypeptide.

In some embodiments, cleavage of the protease motif by a protease in the second cellular environment releases the inhibitory domain from the polypeptide. In some embodiments, release of the inhibitory domain from the target polypeptide allows the target polypeptide to be substantially functional in the second cellular environment. In some embodiments, expression of the protease in the second cellular environment is at least 2 times higher than expression of the protease in the first cellular environment, such as any of about 3, 4, 5, 6, 7, 8, 9, 10, or more, times higher than the expression of the protease in the second cellular environment. In some embodiments, the protease motif is not cleaved by a protease in the first cellular environment. In some embodiments, the inhibitory domain remains fused to the target polypeptide in the first cellular environment. In some embodiments, the target polypeptide function is reduced in the first cellular environment compared to the second cellular environment. In some embodiments, the target polypeptide function is reduced any of about 1-, 2-, 3-, 4-, 5-, 10-fold, or greater, compared to the target polypeptide function in the second cellular environment. In some embodiments, the target polypeptide is inhibited in the first cellular environment. In some embodiments, the first cellular environment is a first species and the second cellular environment is a second species.

In some embodiments, the protease motif is a C1s motif, an elastase motif, a cathepsin motif, a metalloproteinase, a trypsin motif, or a plasminogen activator motif. In some embodiments, the protease is C1s, elastase, cathepsin B/D/G/L/S, trypsin, or plasminogen.

c. Splicing motif

In some aspects, a modulation moiety of the AIR vector is a splicing motif. This embodiment of the AIR vector leverages cell-specific splicing motifs. As shown in FIG. 1C, the splicing motif comprises a 5′-splice site and a 3′-splice site on an IAV mRNA produced from the least one IAV vRNA segment. A region (e.g., intron) between the 5′-splice site and a 3′-splice site is spliced out of the IAV mRNA in specific cellular environments that permits specific splicing of the region (e.g., in cells the recognize the region as a site that should be spliced out of the IAV mRNA). Once the region is spliced out of the IAV mRNA, the IAV mRNA encodes a substantially functional polypeptide of the IAV genome. If the region is not spliced out of the IAV mRNA, the IAV mRNA retains the region (e.g., intron), thereby encoding for a nonsense polypeptide that does not result in a substantially functional polypeptide of the IAV genome.

Thus, in some aspects, provided herein is an AIR vector, wherein the AIR vector comprises at least 7 IAV vRNA segments collectively encoding polypeptides of the IAV genome, wherein at least one IAV vRNA segment of the at least 7 IAV vRNA segments encodes a splicing motif configured such that a target polypeptide of the polypeptides of the IAV genome is not expressed or is inhibited in a first cellular environment, and wherein the target polypeptide is expressed and substantially functional in a second cellular environment. In some embodiments, the splicing motif comprising a 5′-splice site and a 3′-splice site on an IAV mRNA produced from the least one IAV vRNA segment, wherein the IAV mRNA encodes the polypeptide, wherein a region between the 5′-splice site and a 3′-splice site is not spliced out of the splicing motif in the first cellular environment, and wherein the region between the 5′-splice site and a 3′-splice site is spliced out of the splicing motif in the second cellular environment. In some embodiments, the target polypeptide is HA, NA, or a combination thereof.

In some embodiments, the region between the 5′-splice site and a 3′-splice site is not spliced out of the splicing motif in the first cellular environment. In some embodiments, the IAV mRNA comprises an intron when the region between the 5′-splice site and a 3′-splice site is not spliced out of the splicing motif. In some embodiments, when the IAV mRNA comprises the intron, the IAV mRNA contains a nonsense mutation (e.g., the IAV mRNA does not encode a functional polypeptide). In some embodiments, when the IAV mRNA encoding the target polypeptide contains the intron, the target polypeptide is not expressed or the resultant polypeptide is not functional. In some embodiments, expression of the functional target polypeptide is decreased in the first cellular environment when the intron is not spliced out of the splicing motif. In some embodiments, the level of the functional target polypeptide in the first cellular environment is less than about 1%, 2%, 3%, 4%, 5%, 10%, 15%, 20%, 25%, 30%, 35%, 40%, or 50%, compared to level of the functional target polypeptide in the second cellular environment. In some embodiments, the target polypeptide is not expressed in the first cellular environment when the intron is not spliced out of the splicing motif. In some embodiments, the region (e.g., intron) between the 5′-splice site and a 3′-splice site is spliced out of the splicing motif in the second cellular environment. In some embodiments, the IAV mRNA does not comprise the intron when the region between the 5′-splice site and a 3′-splice site is spliced out of the splicing motif. In some embodiments, splicing of the intron out of the splicing motif results in expression of a substantially functional polypeptide of the IAV genome in the second cellular environment. In some embodiments, the first cellular environment is a first species, a first tissue, a first cell, or a first organelle, and the second cellular environment is a second species, a second tissue, a second cell, or a second organelle.

In some embodiments, the splicing motif is on the IAV mRNA produced from the at the least one IAV vRNA segment. In some embodiments, the splicing motif is within an untranslated region (UTR) of the IAV mRNA. In some embodiments, the splicing motif is 5′ to the start codon of the IAV mRNA. In some embodiments, the splicing motif is within the open reading frame (ORF) of the IAV mRNA.

In some embodiments, the splicing motif comprises a sequence derived from the homothorax or Dcam endogenous gene from insects or a flowering locus C (FLC) gene in plants, respectively.

d. IRES Motif

In some aspects, a modulation moiety of the AIR vector is an internal ribosome entry site (IRES) motif. IRES motifs are often used by viruses in place of a 5′ cap modification and/or a polyA tail needed to recruit the ribosome to initiate translation of an mRNA into a polypeptide. This embodiment of the AIR vector leverages cell—and/or species-specific IRES motifs. As shown in FIG. 1B, an IRES motif can be positioned on an IAV mRNA produced from an IAV vRNA segment. The IRES motif recruits a ribosome in a cellular environment where it is recognized, resulting in translation of the IAV mRNA into a polypeptide. If the IRES motif is not recognized, it does not recruit a ribosome, and consequently the IAV mRNA is not translated into a polypeptide. Specifically, the IRES motif may replace a canonical kozak site; the presence of the IRES motif would require translation of the IAV mRNA into a polypeptide. In instances where the IRES motif is not recognized, it may induce a premature stop codon and/or nonsense mediated decay.

Thus, in some aspects, provided herein is an AIR vector, wherein the AIR vector comprises at least 7 IAV vRNA segments collectively encoding polypeptides of the IAV genome, wherein at least one IAV vRNA segment of the at least 7 IAV vRNA segments comprises an IRES motif configured such that a target polypeptide of the polypeptides of the IAV genome is not expressed in a first cellular environment, and wherein the target polypeptide is expressed and substantially functional in a second cellular environment. In some embodiments, the IRES motif is on an IAV mRNA produced from the least one IAV vRNA segment, wherein the IAV mRNA encodes the polypeptide, and wherein the IRES motif recruits a ribosome in the second cellular environment. In some embodiments, the target polypeptide is HA, NA, or a combination thereof.

In some embodiments, the IRES motif does not recruit a ribosome in the first cellular environment. In some embodiments, failure to recruit the ribosome prevents translation of the target polypeptide in the first cellular environment. In some embodiments, failure to recruit the ribosome causes the target polypeptide to be not expressed in the first cellular environment. In some embodiments, the IRES motif recruits a ribosome in the second cellular environment. In some embodiments, the recruiting of the ribosome allows translation of the target polypeptide in the second cellular environment. In some embodiments, the recruiting of the ribosome causes the target polypeptide to be expressed and substantially functional in the second cellular environment. In some embodiments, the first cellular environment is a first species and the second cellular environment is a second species. In some embodiments, the first species and the second species are different species. In some embodiments, the second species is human.

In some embodiments, the IRES motif is on an IAV mRNA produced from the least one IAV vRNA segment, wherein the IAV mRNA encodes the polypeptide. In some embodiments, the IRES motif is 5′ to a translational start site encoding the target polypeptide on the IA VmRNA.

In some embodiments, the IRES motif is from Tobacco Mosiac virus, Barley yellow dwarf virus, Turnip Crinkle virus, Cowpeae mosaic virus, tobacco etch virus, or potato virus A.

iii. Biological Cargo

Also provided herein are AIR vectors comprising a sequence encoding a biological cargo. The sequence encoding a biological cargo (e.g., a foreign gene) may be inserted within a coding region of at least one IAV vRNA segment of the IAV genome.

In some aspects, one or more IAV vRNA segments encodes a biological cargo. Thus, in some embodiments, provided herein is an AIR vector, wherein the AIR vector comprises at least 7 IAV vRNA segments collectively encoding polypeptides of the IAV genome, wherein at least one IAV vRNA segment of the at least 7 IAV vRNA segments encodes a modulation moiety configured such that a target polypeptide of the polypeptides of the IAV genome is not expressed or is inhibited in a first cellular environment, wherein the target polypeptide is expressed and substantially functional in a second cellular environment, and wherein one or more IAV vRNA segments of the at least 7 vRNA segments encodes a biological cargo.

In some embodiments, the biological cargo is encoded by any of the IAV vRNA segments of the at least 7 IAV vRNA segments. In some embodiments, the biological cargo is encoded by IAV vRNA segment 1. In some embodiments, the biological cargo is encoded by IAV vRNA segment 2. In some embodiments, the biological cargo is encoded by IAV vRNA segment 3. In some embodiments, the biological cargo is encoded by IAV vRNA segment 4. In some embodiments, the biological cargo is encoded by IAV vRNA segment 5. In some embodiments, the biological cargo is encoded by IAV vRNA segment 6. In some embodiments, the biological cargo is encoded by IAV vRNA segment 8. In some embodiments, the biological cargo is not encoded by IAV vRNA segment 7. In some embodiments, the at least 7 vRNA segments comprise IAV vRNA segment 1 encoding PB2 and NS1, IAV vRNA segment 2 encoding PB1 and NS2, IAV vRNA segment 3 encoding PA, IAV vRNA segment 4 encoding HA, IAV vRNA segment 5 encoding NP, IAV vRNA segment 6 encoding NA, IAV vRNA segment 7 encoding M1 and M2, and IAV vRNA segment 8 encoding a biological cargo.

In some embodiments, the sequence encoding a biological cargo comprises between about 200 and about 7,000 nucleotides, such as between any of about 200 nucleotides and about 500 nucleotides, about 400 nucleotides and about 1,000 nucleotides, about 500 nucleotides and about 3,000 nucleotides, about 2,000 nucleotides and about 4,000 nucleotides, and about 3,000 nucleotides and about 7,000 nucleotides. In some embodiments, the sequence encoding biological cargo comprises less than about 7,000 nucleotides, such as less than any of about 7,000, 6,500, 6,000, 5,500, 5,000, 4,500, 4,000, 3,500, 3,000, 2,500, 2,400, 2,300, 2,200, 2,100, 2,000, 1,900, 1,800, 1,700, 1,600, 1,500, 1,400, 1,300, 1,200, 1,100, 1,000, 900, 800, 700, 600, 500, 400, 300, 200, or fewer, nucleotides. In some embodiments, the sequence encoding biological cargo comprises greater than about 200 nucleotides, such as greater than any of about 300, 400, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 3,000, 3,500, 4,000, 4,500, 5,000, 5,500, 6,000, 6,500, 7,000, or more, nucleotides. In some embodiments, the sequence encoding biological cargo comprises up to about 1,500 when the biological cargo is encoded by IAV vRNA segment 1. In some embodiments, the sequence encoding biological cargo comprises up to about 1,000 when the biological cargo is encoded by IAV vRNA segment 2. In some embodiments, the sequence encoding biological cargo comprises up to about 2,300 when the biological cargo is encoded by IAV vRNA segment 3. In some embodiments, the sequence encoding biological cargo comprises up to about 700 when the biological cargo is encoded by IAV vRNA segment 4. In some embodiments, the sequence encoding biological cargo comprises up to about 900 when the biological cargo is encoded by IA V vRNA segment 5. In some embodiments, the sequence encoding biological cargo comprises up to about 1,000 when the biological cargo is encoded by IAV vRNA segment 6. In some embodiments, the sequence encoding biological cargo comprises up to about 1,000 when the biological cargo is encoded by IAV vRNA segment 8.

In some embodiments, the biological cargo is a coding RNA or a non-coding RNA. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the antigen induces immunity against a pathogen in the first cellular environment. In some embodiments, the biologically active polypeptide replaces the function of a defective gene in the first cellular environment.

In some embodiments, one or more IAV vRNA segments of the at least 7 IAV vRNA segments collectively encoding polypeptides of the IAV genome encodes a self-cleaving peptide to generate a polycistronic mRNA. In some embodiments, the ribosomal skipping peptide encoded by the one or more IAV segments is 2A ribosomal skipping peptide (e.g., porcine teschovirus-1 2A (P2A), Thosea asigna virus 2A (T2A), equine rhinitis A virus 2A (E2A), or foot-and-mouth disease virus 2A (F2A)). Thus, in some embodiments, one or more IAV vRNA segments of the at least 7 IAV vRNA segments collectively encoding polypeptides of the IAV genome encodes a 2A ribosomal skipping peptide (e.g., P2A, T2A, E2A, F2A). In some embodiments, a 2A ribosomal skipping peptide (e.g., P2A, T2A, E2A, F2A) is encoded on the same IAV vRNA segment as the biological cargo. In some embodiments, a 2A ribosomal skipping peptide (e.g., P2A, T2A, E2A, F2A) is encoded between the sequence encoding the biological cargo and a region of the IAV vRNA segment encoding a polypeptide of the IAV genome. In some embodiments, a 2A ribosomal skipping peptide (e.g., P2A, T2A, E2A, F2A) is encoded between two regions of an IAV vRNA segment encoding two polypeptides of the IAV genome.

In some embodiments, one or more IAV vRNA segments of the at least 7 IAV vRNA segments collectively encoding polypeptides of the IAV genome encodes a ribosomal skipping peptide and a Cre recombinase. In some embodiments, the ribosomal skipping peptide encoded by the one or more IAV segments is a 2A ribosomal skipping peptide (e.g., P2A, T2A, E2A, F2A). In some embodiments, the 2A ribosomal skipping peptide (e.g., P2A, T2A, E2A, F2A) is encoded on the same IAV vRNA segment as the Cre recombinase. In some embodiments, the 2A ribosomal skipping peptide (e.g., P2A, T2A, E2A, F2A) is encoded between the sequence encoding the Cre recombinase and a region of the IAV vRNA segment encoding a polypeptide of the IAV genome.

### C. Pharmaceutical Compositions and Kits

i. Pharmaceutical Compositions

The AIR vector may be formulated into various compositions for use in various methods (e.g., gene delivery, therapeutic, and/or prophylactic treatment methods). In particular, the AIR vector can be made into a pharmaceutical composition by combination with appropriate pharmaceutically acceptable carriers or diluents and can be formulated to be appropriate for either human or veterinary applications.

Thus, in some aspects, provided herein is a pharmaceutical composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes a modulation moiety configured such that a target polypeptide of the polypeptides of the IAV genome is not expressed or is inhibited in a first cellular environment, and wherein the target polypeptide is expressed and substantially functional in a second cellular environment, and a pharmaceutically acceptable carrier. As used herein, a “pharmaceutically acceptable carrier” refers to a substrate, composition, or vehicle in the process of AIR vector delivery, which may have one or more ingredients including, but not limited to, excipient(s), binder(s), diluent(s), solvent(s), filler(s), and/or stabilizer(s). A variety of pharmaceutically acceptable carriers can be used that are suitable for administration. The choice of carrier will be determined, in part, by the particular features of the AIR vector, as well as by the particular method used to administer the pharmaceutical composition.

A pharmaceutical composition comprising the provided AIR vector, alone or in combination with other antiviral compounds, can be made into a formulation suitable for administration intravenously, intranasally, and/or intrabronchially (e.g., orally). Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be provided in unit dose or multidose sealed containers, such as ampules and vials, and can be stored in a freeze-dried (lyophilized) condition requiring only the addition of the sterile liquid carrier, for example, water, for injections, immediately prior to use. Injectable solutions and suspensions can be prepared from sterile powders, granules, and tablets, as described herein.

In some embodiments, the formulation is an aerosol formulation suitable for administration via inhalation. The aerosol formulation can be placed into a pressurized acceptable propellant, such as dichlorodifluoromethane, propane, nitrogen, and the like.

In some embodiments, the formulation is an aerosol formulation suitable for oral administration. A formulation suitable for oral administration can be a liquid solution, such as an effective amount of a subject interfering construct or a subject interfering particle dissolved in diluents, such as water, saline, or fruit juice; capsules, sachets or tablets, each containing a predetermined amount of the active agent (a subject interfering construct or subject interfering particle), as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

In some embodiments, there is provided a pharmaceutical composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes an MSE configured such that a target polypeptide of the polypeptides of the IAV genome is not expressed or is inhibited in a first cellular environment, and wherein the target polypeptide is expressed and substantially functional in a second cellular environment, and a pharmaceutically acceptable carrier. In some embodiments, the MSE is on an IAV mRNA produced from the least one IAV vRNA segment, the IAV mRNA encodes the polypeptide, and the MSE is targeted by a miRNA in the first cellular environment. In some embodiments, the miRNA silences expression of the target polypeptide in the first cellular environment. In some embodiments, the miRNA is not expressed in the second cellular environment. In some embodiments, the MSE is on the IAV mRNA produced from the at least one IAV vRNA segment. In some embodiments, the MSE is within an untranslated region (UTR) (e.g., 5′ UTR or 3′ UTR) of the IAV mRNA. In some embodiments, the MSE is 5′ to the start codon of the IAV mRNA. In some embodiments, the MSE is 3′ to the stop codon of the IAV mRNA. In some embodiments, the MSE is within the open reading frame (ORF) of the IAV mRNA. In some embodiments, the first cellular environment is a first species, a first tissue, a first cell, or a first organelle, and the second cellular environment is a second species, a second tissue, a second cell, or a second organelle. In some embodiments, one or more IAV vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In some embodiments, there is provided a pharmaceutical composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes an inhibitory domain configured such that a target polypeptide of the polypeptides of the IAV genome is not expressed or is inhibited in a first cellular environment, and wherein the target polypeptide is expressed and substantially functional in a second cellular environment, and a pharmaceutically acceptable carrier. In some embodiments, the inhibitory domain is encoded within the ORF of an IAV mRNA produced from the at least one vRNA segment, wherein the IAV mRNA encodes the target polypeptide. In some embodiments, cleavage of the protease motif by a protease in the second cellular environment releases the inhibitory domain from the target polypeptide. In some embodiments, the target polypeptide is inhibited in the first cellular environment. In some embodiments, the first cellular environment is a first species and the second cellular environment is a second species. In some embodiments, one or more IAV vRNA segments of the at least 7 IAV vRNA segments encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof . . .

In some embodiments, there is provided a pharmaceutical composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes a splicing motif configured such that a target polypeptide of the polypeptides of the IAV genome is not expressed or is inhibited in a first cellular environment, and wherein the target polypeptide is expressed and substantially functional in a second cellular environment, and a pharmaceutically acceptable carrier. In some embodiments, the splicing motif comprising a 5′-splice site and a 3′-splice site on an IAV mRNA produced from the least one IAV vRNA segment, wherein the IAV mRNA encodes the polypeptide, wherein a region between the 5′-splice site and a 3′-splice site is not spliced out of the splicing motif in the first cellular environment, and wherein the region between the 5′-splice site and a 3′-splice site is spliced out of the splicing motif in the second cellular environment. In some embodiments, the region between the 5′-splice site and a 3′-splice site is not spliced out of the splicing motif in the first cellular environment. In some embodiments, the splicing motif is within the open reading frame (ORF) of the IAV mRNA. In some embodiments, one or more IAV vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In some embodiments, there is provided a pharmaceutical composition comprising an AIR vector, wherein the AIR vector comprises at least 7 IAV vRNA segments collectively encoding polypeptides of the IAV genome, wherein at least one IAV vRNA segment of the at least 7 IAV vRNA segments comprises an IRES motif configured such that a target polypeptide of the polypeptides of the IAV genome is not expressed in a first cellular environment, and wherein the target polypeptide is expressed and substantially functional in a second cellular environment, and a pharmaceutically acceptable carrier. In some embodiments, the IRES motif is on an IAV mRNA produced from the least one IAV vRNA segment, wherein the IAV mRNA encodes the polypeptide, and wherein the IRES motif recruits a ribosome in the second cellular environment. In some embodiments, the first cellular environment is a first species and the second cellular environment is a second species. In some embodiments, the first species and the second species are different species. In some embodiments, the first species is human. In some embodiments, the IRES motif is 5′ to a translational start site of the ORF encoding the target polypeptide on the IAV mRNA. In some embodiments, one or more IAV vRNA segments of the at least 7 IAV vRNA segments collectively encoding polypeptides of the IAV genome encodes a biological cargo. In some embodiments, the biological cargo is an antigen, a biologically active polypeptide (e.g., cytokine), a ncRNA, a marker polypeptide (e.g., GFP), one or more components of a gene editing system, or a Cre recombinase. In some embodiments, the target polypeptide is HA, NA, or a combination thereof.

In some embodiments, there is provided a pharmaceutical composition comprising a composition comprising an AIR vector, wherein the AIR vector comprises at least 7 IAV vRNA segments collectively encoding polypeptides of the IAV genome, wherein at least one IAV vRNA segment of the at least 7 IAV vRNA segments encodes a modulation moiety configured such that a target polypeptide of the polypeptides of the IAV genome is not expressed or is inhibited in a first cellular environment, wherein the target polypeptide is expressed and substantially functional in a second cellular environment, and wherein one or more IAV vRNA segments of the at least 7 IAV vRNA segments collectively encoding polypeptides of the IAV genome encodes a biological cargo, and a pharmaceutically acceptable carrier. In some embodiments, the modulation moiety is an MSE, an inhibitory domain, a splicing motif, or an IRES motif. In some embodiments, the target polypeptide is HA, NA, or a combination thereof.

ii. Kits

Kits are described herein that include unit doses of an AIR vector, such as any of the AIR vectors provided herein. The unit doses can be formulated for nasal, oral, transdermal, or injectable (e.g., for intramuscular, intravenous, or subcutaneous injection) administration. In such kits, in addition to the containers containing the unit doses will be an informational package insert describing the use of the provided AIR vector. Thus, in some aspects, provided herein is a kit for use of a composition, or a pharmaceutical composition, comprising an AIR vector, such as any of the AIR vectors provided herein.

In some embodiments, the kit comprises instructions for practicing the methods of use provided herein, or means for obtaining the same (e.g., a website URL directing the user to a webpage which provides the instructions). In some embodiments, the instructions are printed on a substrate, such as a package insert, the packaging, formulation containers, and the like.

### D. Methods of Use

Various applications and uses of the provided AIR vector are contemplated herein. For example, in some embodiments, the methods provided herein utilize the provided AIR vectors for gene therapy, cell therapy, and/or treatment or prevention of a disease.

In some aspects, provided herein is method of treating or preventing a disease or disorder associated in an individual comprising administering a composition comprising an AIR vector, such as any of the AIR vectors provided herein, or a pharmaceutical composition comprising an AIR vector, such as any of the AIR vectors provided herein, and a pharmaceutically acceptable carrier, to an individual. In some aspects, one or more IAV vRNA segments of the at least 7 IAV vRNA segments collectively encoding polypeptides of the IAV genome encodes a biological cargo. In some embodiments, the disease is a viral infection, an immune disease, a cancer, a disease treatable by an enzyme replacement therapy, or a disease treatable by gene editing. In some embodiments, the viral infection is from a virus is of the Orthomyxoviridae family. In some embodiments, the virus is an IAV. In some embodiments, the individual has been exposed to the virus. In some embodiments, the individual is prone to infection by the virus (e.g., an elderly individual, an infant, or an immunocompromised individual). In some embodiments, the cancer is non-small cell lung cancer (NSCLC), including adenocarcinoma, squamous cell carcinoma, and large cell carcinoma. In some embodiments, the cancer is small cell lung cancer (SCLC). In some embodiments, the cancer is bladder cancer, such as non-muscle-invasive bladder cancer (NMIBC). In some embodiments, the disease benefits from a targeted therapy. In some embodiments, the AIR vector comprises a sequence encoding a biological cargo. In some embodiments, one or more IAV vRNA segments of at least 7 IAV vRNA segments collectively encoding polypeptides of the IAV genome (e.g., one or more IAV vRNA segments encodes a biological cargo. In some embodiments, the AIR vector (e.g., a composition comprising the AIR vector, e.g., a pharmaceutical composition comprising the AIR vector) is administered by intramuscular injection, intranasal administration, intravenous infection, or inhalation administration. In some embodiments, the individual is a human.

In some embodiments, provided herein is a method of delivering a gene to an individual, the method comprising administering a composition comprising an AIR vector, such as any of the AIR vectors provided herein, or a pharmaceutical composition comprising an AIR vector, such as any of the AIR vectors provided herein, and a pharmaceutically acceptable carrier, to the individual. In some embodiments, the AIR vector comprises the gene (e.g., a biological cargo). In some embodiments, one or more IAV vRNA segments of at least 7 IAV vRNA segments collectively encoding polypeptides of the IAV genome (e.g., one or more IAV vRNA segments encodes the gene. In some embodiments, the individual comprises a defective version of the gene (e.g., the gene comprises a mutation). In some embodiments, the individual does not comprise the gene. In some embodiments, the AIR vector (e.g., a composition comprising the AIR vector, e.g., a pharmaceutical composition comprising the AIR vector) is administered by intramuscular injection, intranasal administration, intravenous infection, or inhalation administration. In some embodiments, the individual is a human.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is suspected of having a disease (e.g., viral infection, an immune disease, a cancer, a disease treatable by an enzyme replacement therapy, or a disease treatable by gene editing). In some embodiments, the individual is diagnosed with the disease (e.g., viral infection, an immune disease, a cancer, a disease treatable by an enzyme replacement therapy, or a disease treatable by gene editing). In some embodiments, the individual is diagnosed with a viral infection, an immune disease, a cancer, a disease treatable by an enzyme replacement therapy, or a disease treatable by gene editing. In some embodiments, the individual is diagnosed with a viral infection caused by a virus of the Orthomyxoviridae family. In some embodiments, the individual is diagnosed with influenza, such as IAV. In some embodiments, the individual has not been exposed to the virus of the Orthomyxoviridae family. In some embodiments, the individual is diagnosed with a cancer. In some embodiments, the individual is diagnosed with an immune disease. In some embodiments, the cancer is non-small cell lung cancer (NSCLC), including adenocarcinoma, squamous cell carcinoma, and large cell carcinoma. In some embodiments, the cancer is small cell lung cancer (SCLC). In some embodiments, the cancer is bladder cancer, such as non-muscle-invasive bladder cancer (NMIBC).

Efficacy of the methods of treatment or prevention can be evaluated, for example, by evaluating viral load (e.g., via detection of viral DNA), duration of survival of the individual, quality of life of the individual, viral protein expression and/or activity, and/or detection of serological antibodies against the virus of the Orthomyxoviridae family. Efficacy of the methods of delivering a gene can be evaluated by, for example, duration of survival of the individual, quality of life of the individual, and/or gene expression and/or activity.

In some embodiments, provided herein is a method of delivering a gene to a cell comprising administering a composition comprising an AIR vector, such as any of the AIR vectors provided herein, or a pharmaceutical composition comprising an AIR vector, such as any of the AIR vectors provided herein, and a pharmaceutically acceptable carrier, to the cell. In some embodiments, the AIR vector is not capable of replicating in the cell. In some embodiments, the AIR vector comprises a sequence encoding a biological cargo. In some embodiments, one or more IAV vRNA segments of at least 7 IAV vRNA segments collectively encoding polypeptides of the IAV genome (e.g., one or more IAV vRNA segments encodes a biological cargo.

In some embodiments, the cell is an airway cell.

In some embodiments, when the IAV vector is a pseudotype virus vector (e.g., with either a vesiculovirus or thogotovirus G receptor), the virus may enter any cell type.

In some embodiments, provided herein is a method of producing an AIR vector, such as any of the AIR vectors provided herein, in a second cellular environment. In some embodiments, a target polypeptide of the polypeptides of the IAV genome is not expressed or is inhibited in a first cellular environment, and wherein the target polypeptide is expressed and substantially functional in the second cellular environment. In some embodiments, the AIR vector is capable of replicating in the second cellular environment. In some embodiments, introducing one or more polynucleotides encoding said vRNA segments of the AIR vector to the second cellular environment allows for replication of the AIR vector.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Generation of an AIR Vector with a miRNA Cell-Specific Modulation Motif

This Example demonstrates the generation of an AIR vector comprising a miRNA modulation motif which confines translation of HA and/or NA transcripts to in ovo setting.

The unique miRNA environment in the allantois of fertilized chicken eggs was used to produce an IAV virus. Briefly, a conditional AIR vector was engineered to selectively interrupt translation of specific transcripts in all but an in ovo environment by employing miRNAs that are ubiquitous in developed vertebrate animals, but absent in cells comprising the allantois.

miRNAs such as miR-130b, miR-200a, miR-148a, miR-215, miR-363, miR-1552, miR-2188, and miR-6635 comprise the vast majority of small RNAs in the allantois. See, e.g., Wade, B. et al. (2016) Isolation and detection of microRNA from the egg of chickens. BMC Res Notes 9, 283; Zhou, K. Z. et al. (2022) Comparative Analysis of miRNA Expression Profiles in Skeletal Muscle of Bian Chickens at Different Embryonic Ages. Animals (Basel) 12; Shi, J. et al. (2022) MiRNA sequencing of Embryonic Myogenesis in Chengkou Mountain Chicken. BMC Genomics 23, 571. In contrast, any combination of miRNAs such as miR-21, miR-31, miR-192, miR-93, and miR-29b can be readily detected in all cells of a developed vertebrate. See, e.g., Benitez, A. A. et al. (2015) Engineered Mammalian RNAi Can Elicit Antiviral Protection that Negates the Requirement for the Interferon Response. Cell Rep 13, 1456-1466; Nilsson-Payant, B. E. et al. (2021) Reduced Nucleoprotein Availability Impairs Negative-Sense RNA Virus Replication and Promotes Host Recognition. J Virol 95. As a result, engrafting perfect target sites, corresponding to these five ubiquitous miRNAs that are not represented in the allantois, as a miRNA silencing element (MSE), confines expression of the associated transcript to an in ovo setting.

Inserting this miRNA silencing element into HA and NA vRNA segments, for example, resulted in an IAV vector which maintains replicative capacity in a fertilized chicken egg while generating an AIR incapable of replication in differentiated vertebrate cells due to the absence of the viral HA and NA glycoproteins.

### Example 2. Characterization of the AIR Vector

This Example demonstrates that an exemplary AIR vector, as described in Example 1, only produces functional HA and NA protein in ovo to allow for IAV replication.

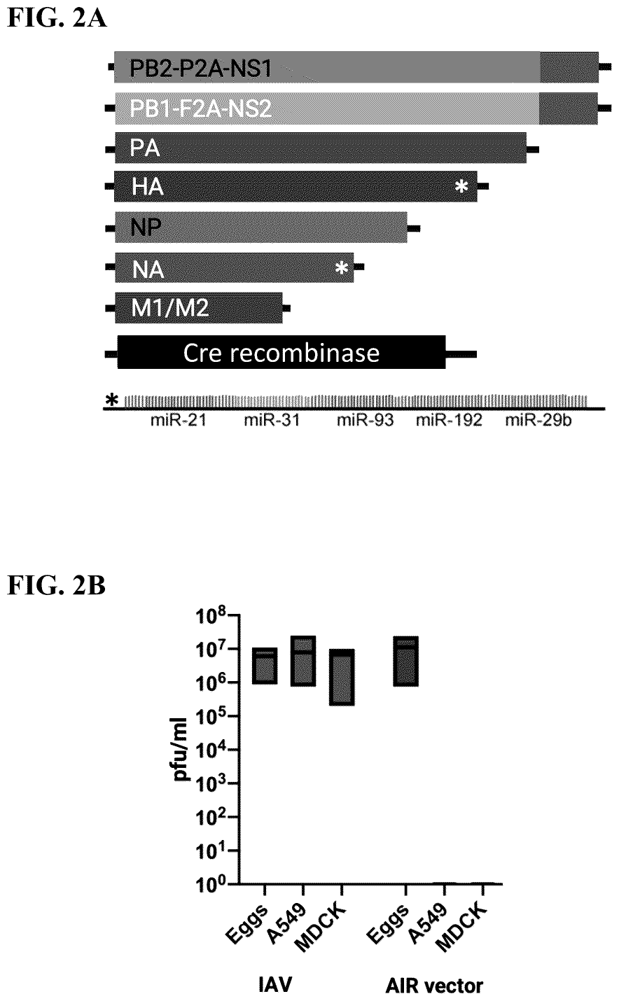

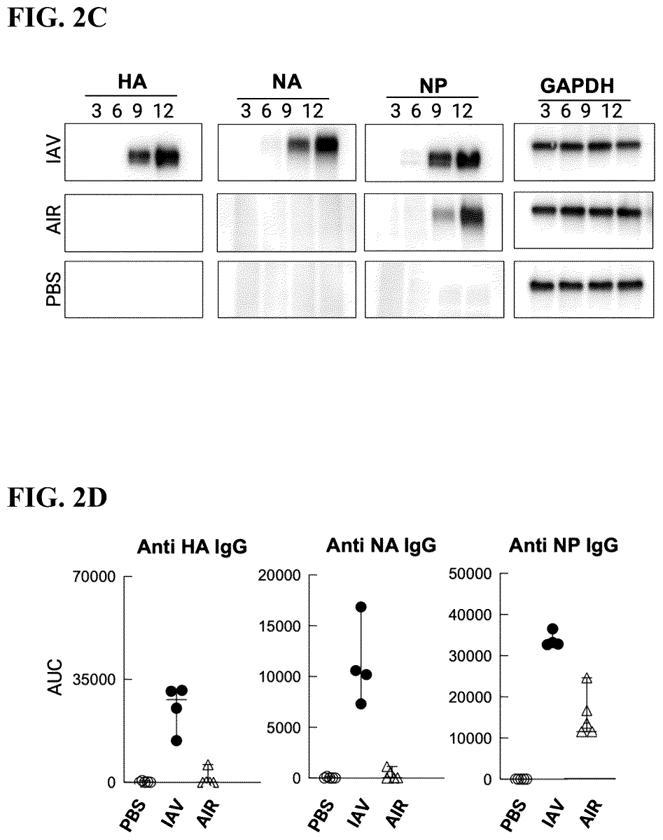

An exemplary AIR vector of the present invention is depicted in FIG. 2A. In this construct, the NS1 and NS2 genes were moved to segment one (PB2-P2A-NS1) and two (PB1-F2A-NS2). In addition, a miRNA targeting cassette containing miR-21, miR-31, miR-93, miR-192, and miR-29b targets was inserted into the 3′ UTR of the HA and NA genes. The miRNA target sequences are the reverse complement of the miRNA sequences provided below

miR-21-5p:
(SEQ ID NO: 1)
UAGCUUAUCAGACUGAUGUUGA;
miR-31-5p:
(SEQ ID NO: 2)
AGGCAAGAUGCUGGCAUAGCU;
miR-192-5p:
(SEQ ID NO: 3)
CUGACCUAUGAAUUGACAGCC;
miR-93-5p:
(SEQ ID NO: 4)
CAAAGUGCUGUUCGUGCAGGUAG;
miR-29b-3p:
(SEQ ID NO: 5)
UAGCACCAUUUGAAAUCAGUGUU.

This construct enables delivery of a cargo (e.g., GFP, Cre recombinase, etc.) from segment eight (FIG. 2A). Genetic rescue of this virus demonstrated wild type levels of replication in ovo (eggs) with no replication observed in either human lung epithelial cells (A549) or in canine kidney cells (MDCK) as expected (FIG. 2B). When IAV wild type was compared to the AIR platform in mammalian cells, HA and NA protein could not be detected despite having comparable levels of NP (FIG. 2C). Moreover, when administered repeatedly to mice over a three-week period, no antibodies to HA or NA were observed, while an IgG response to NP was noted (FIG. 2D).

These results indicate that IAV produced by the AIR vector is unable to replicate in mammalian cells but successfully replicates in ovo.

### Example 3. In Vivo Characterization of the AIR Vector in Mice

This Example demonstrates that in vivo administration of the AIR vector, as described in Examples 1 and 2, results in cell-specific expression of IAV in mice.

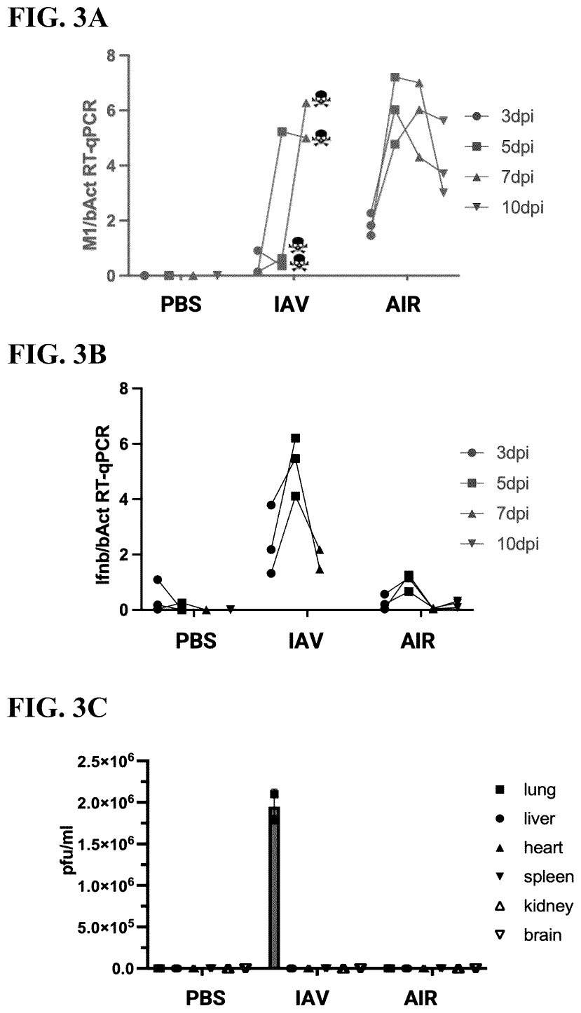

Intranasal administration of the AIR vector (10\hat{ }4 pfu) or wild type IAV (A/Puerto Rico/04/1938, 50pfu) to mice were next assessed 3-, 5-, 7-, and 10-days post treatment. Viral transcript levels were comparable between AIR vector and wild type IAV, but IAV proved to be lethal whereas no morbidity or mortality was observed when using the AIR platform. Expression of M1 was found to peak at 5-days post treatment, diminishing by day 10 (FIG. 3A). Assessing engagement of the host innate defenses via IFNβ transcripts also showed that while IAV induced robust levels of IFNβ during infection, the AIR platform was comparable to phosphate buffered saline (PBS) controls (FIG. 3B).

Lastly, necropsy was performed on mice infected with IAV or treated with AIR to assess infectivity of different organs. The distribution of IAV infectious material was limited to the lungs as no other tissues yielded plaque forming units (FIG. 3C). In contrast, as AIR was unable to generate HA nor NA beyond the allantois cellular environment, tittering of organs obtained from necropsy showed no infectious AIR material in any organ, including the lungs (FIG. 3C).

These data show that targeting HA and NA by inserting a miRNA silencing element comprising target sites for miR-21, miR-31, miR-192, miR-93, and miR-29b is sufficient to generate a vector that can express exogenous transcripts for 7-10 days in vivo in the absence of immune engagement.

### Example 4. In Vivo Characterization of the AIR Vector in Ferrets

This Example demonstrates that in vivo administration of the AIR vector, as described in Examples 1 and 2, results in cell-specific expression of IAV in ferrets.

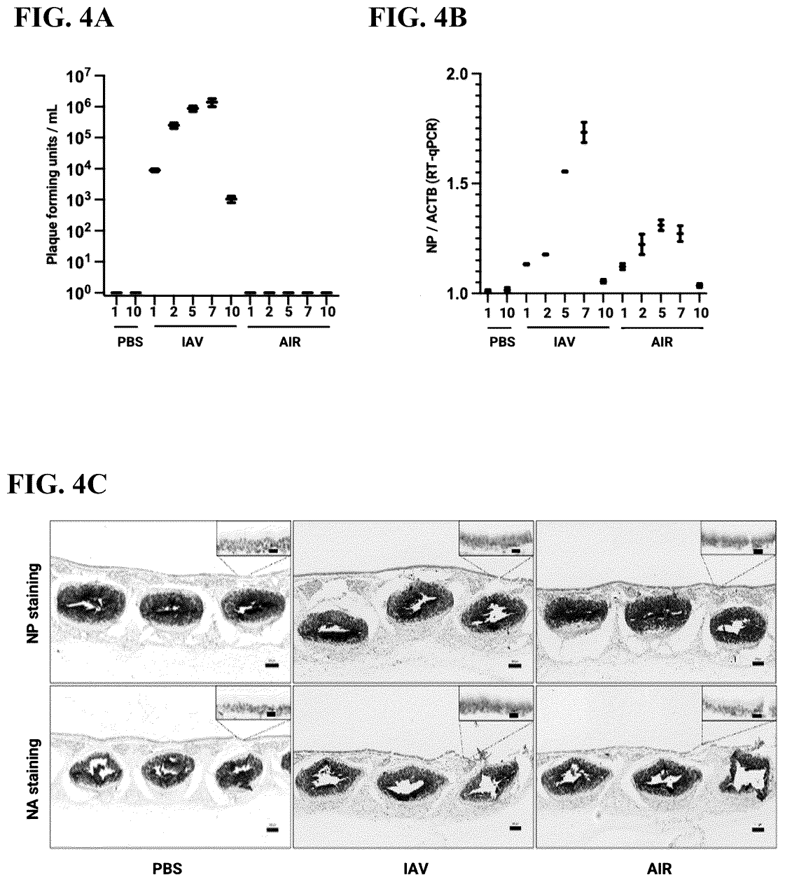

Treatment of IAV (1000 pfu IN) resulted in shedding of nasal washes as early as one day post infection, peaked on day seven, and diminished thereafter in ferrets treated as in Example 3 (FIG. 4A). In contrast, administration of the AIR vector (10\hat{ }7 pfu IN) resulted in no viral shedding at any time point (FIG. 4A). Similarly to what was observed in mice, the analysis of NP levels from IAV challenge and AIR-treated ferrets demonstrated a similar expression profile (Example 3). In IAV administration, NP levels were detected as early as one day post challenge, peaking on day seven, and diminishing on day ten (FIG. 4B). This same analysis following the administration of the AIR vector showed detection of NP at one day post infection, peaking at five days post infection, and diminishing to baseline values by ten days post treatment (FIG. 4B).

Immunohistochemistry of the trachea in these animals was subsequently performed to assess levels of NP and NA. These data found that ciliated cells in the trachea stained uniformly positive for both NP and NA following IAV challenge at three days post infection in contrast to the AIR vector which only generated NP positive cells, corroborating in vitro data (FIG. 4C).

These data show that targeting HA and NA by inserting a miRNA silencing element comprising target sites for miR-21, miR-31, miR-192, miR-93, and miR-29b is sufficient to generate a vector that can express exogenous transcripts for 7-10 days in vivo in the absence of immune engagement.

### Example 5. In Vivo Biological Cargo Delivery Using an AIR Vector

This Example demonstrates that an exemplary AIR vector can be successfully used to deliver a biological cargo in mice.

To demonstrate the delivery potential of the AIR vector, a Cre recombinase was embedded into segment eight as the cargo of interest (FIG. 2A). This construct, denoted as AIR-Cre, was administered to Ai9 mice as described in Madisen, L. et al. (2010), A robust and high-throughput Cre reporting and characterization system for the whole mouse brain. Nat Neurosci 13, 133-140. This strain of mice comprises a Cre reporter as they encode a loxP-flanked stop cassette preventing transcription of a CAG promoter-driven red fluorescent protein variant (tdTomato)-all inserted into the Gt (ROSA) 26Sor locus. Ai9 mice express robust tdTomato fluorescence following Cre-mediated recombination. As such, 10⁴ pfu of AIR or AIR-Cre was administered to Ai9 mice and mice were euthanized seven days post treatment. Lungs and trachea were isolated, cryopreserved, sectioned, and used to assess tdTomato fluorescence thereafter.

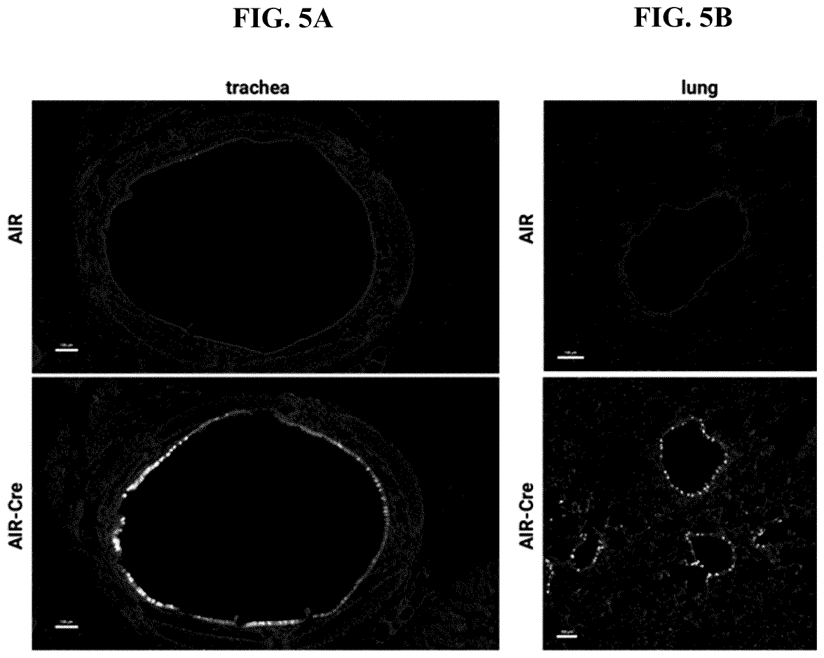

Analysis of the trachea showed extensive tdTomato expression in exposed ciliated cells uniquely in animals treated with AIR-Cre (FIG. 5A). Similarly, analysis of the lungs showed positive tdTomato staining in the large airways in response to AIR-Cre (FIG. 5B). Taken together, these data demonstrate effective delivery of a biological cargo (Cre recombinase) using the AIR platform.

### Example 6. Effective Biological Cargo Delivery Using Alternative AIR Vectors

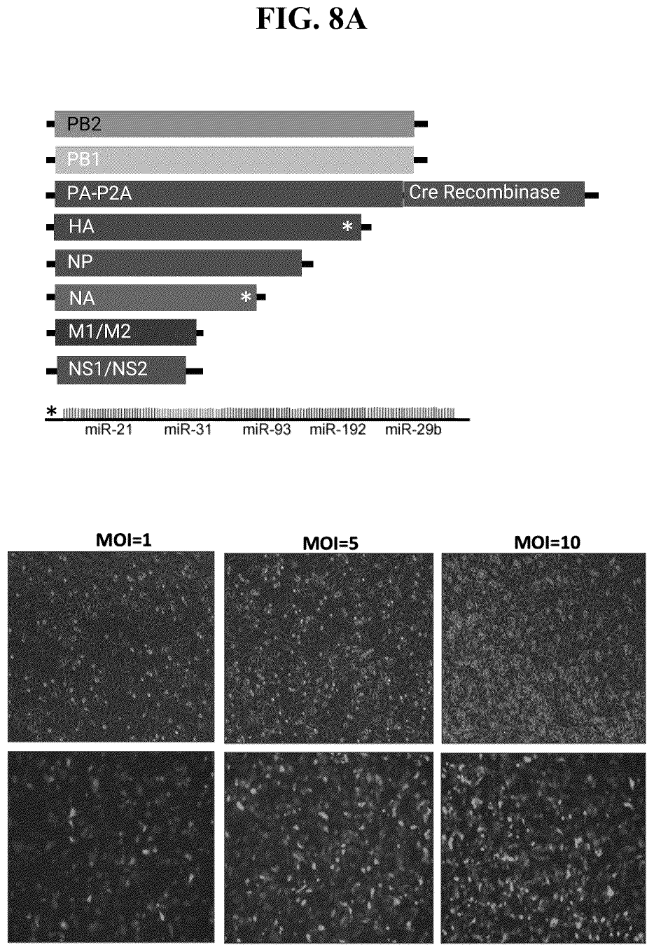

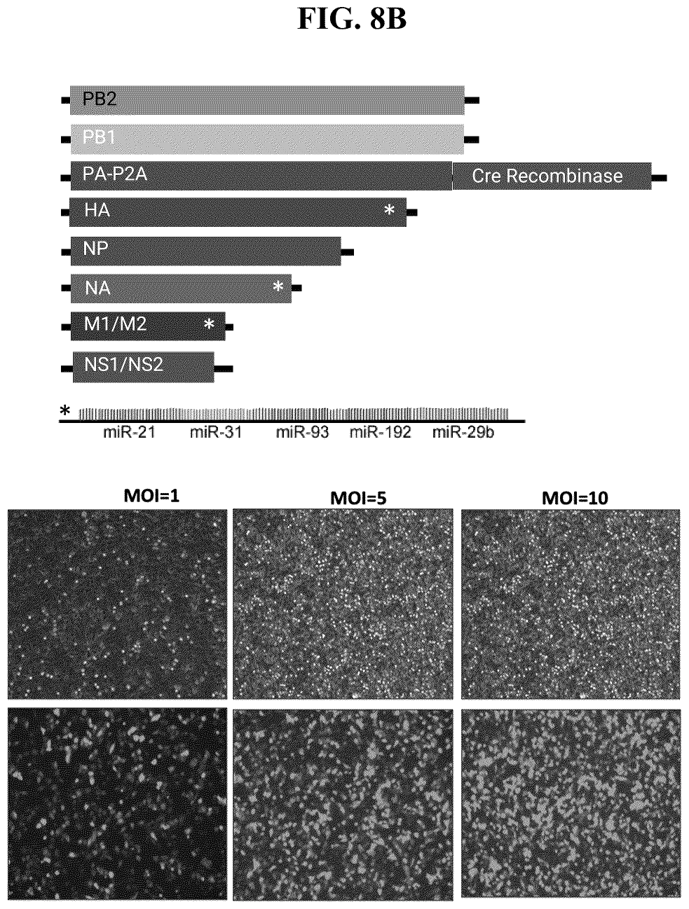

Two alternative AIR vectors were designed to deliver a biological cargo (Cre recombinase) in a reporter assay. FIG. 8A shows a schematic of an AIR design where segment 3 is used to encode a cargo and miRNA Silencing Elements are introduced to the segments encoding HA and NA. FIG. 8B shows a schematic of another AIR design similar to FIG. 8A but the miRNA Silencing Elements are additionally introduced into the segment encoding M1/M2. The AIR vectors were used to transduce human lung epithelial cells (A549) Cre-reporter cells. The A549 cells encode a loxP-flanked stop cassette preventing transcription of a Green fluorescent protein (GFP). Upon Cre delivery and excision of the loxP-flanked cassette, A549 cells can express robust GFP fluorescence (FIGS. 8A-8B). Additional targeting of M1/M2 resulted in a modest increase of cargo expression.

### Example 7. In Vivo and In Vitro Delivery of Cytokines Using Alternative AIR Vectors

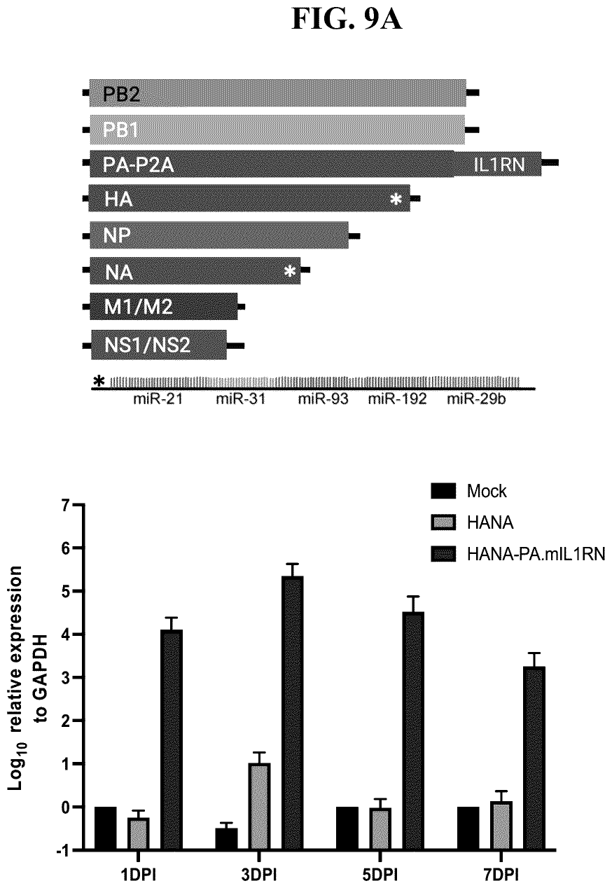

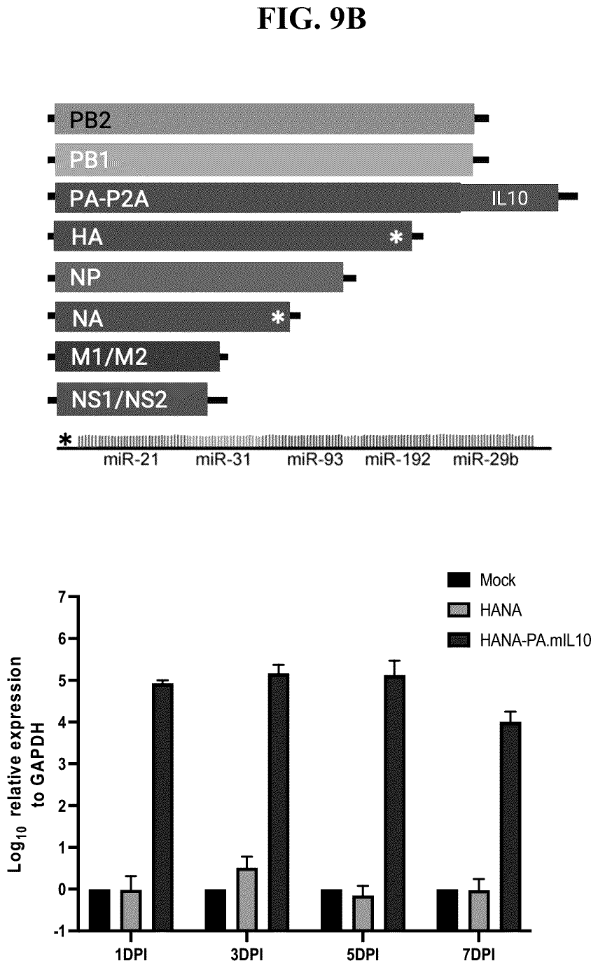

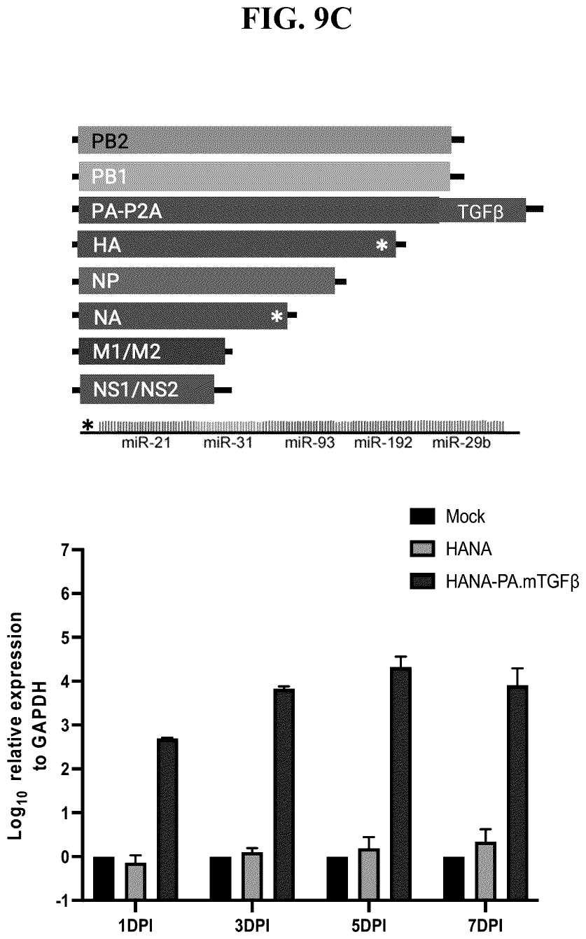

In this Example, an HA/NA miRNA targeted AIR vector was used to deliver different cargos in vivo. The designs of the AIR vector are depicted in FIGS. 9A-9C. A cytokine, interleukin 1 receptor antagonist (ILIRN) (FIG. 9A), interleukin 10 (IL 10) (FIG. 9B), or transforming growth factor beta (TGF-β) (FIG. 9C), was encoded in segment 3 of the respective vectors. Cohorts of C57BL/6 mice (n=5/cohort) were treated with 10\hat{ }4 pfu of the AIR vector. Expression of the cytokines was measured by RT-qPCR from lung tissues of the treated mice at the indicated days post infection (DPI). The results demonstrated that the AIR vector was able to successfully deliver different cytokines in vivo.

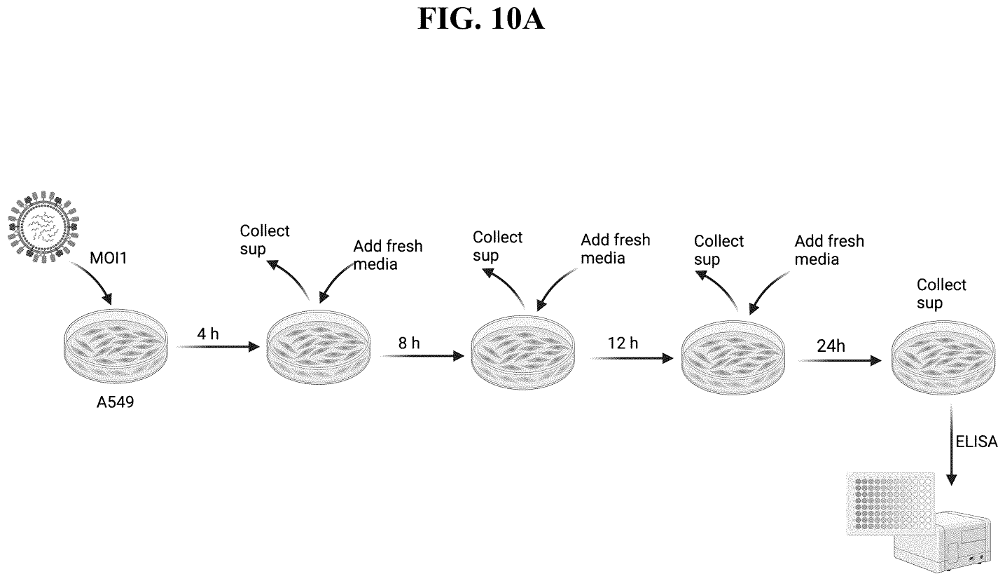

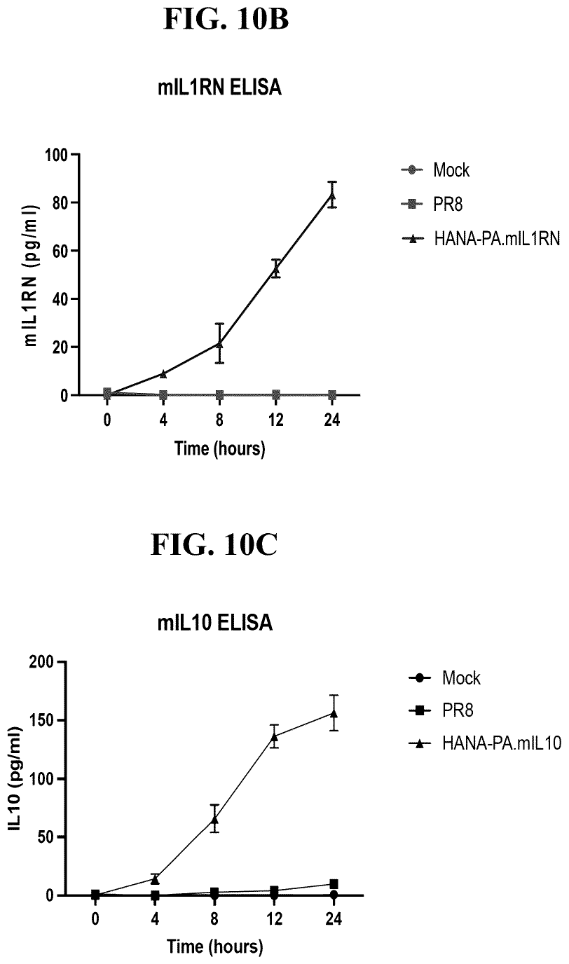

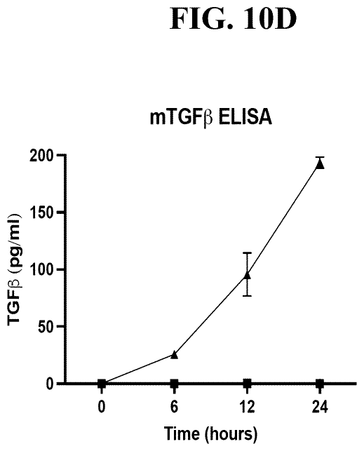

The same HA/NA miRNA targeted AIR vectors were then tested in an in vitro assay to assess whether the encoded cytokines were properly secreted and recognized. A549 cells were treated with the AIR vectors at an MOI of 1. Supernatants were collected from the cells according to the study schedule described in FIG. 10A. An enzyme linked immunosorbent assay (ELISA) was used to measure levels of the respective cytokines, ILIRN, IL10, and TGF-β, in the supernatants collected at the indicted time points post AIR vector treatment. The results (FIG. 10B-10D) demonstrated that all encoded cytokines were secreted and recognized, suggesting that the cytokines folded properly after AIR vector delivery.

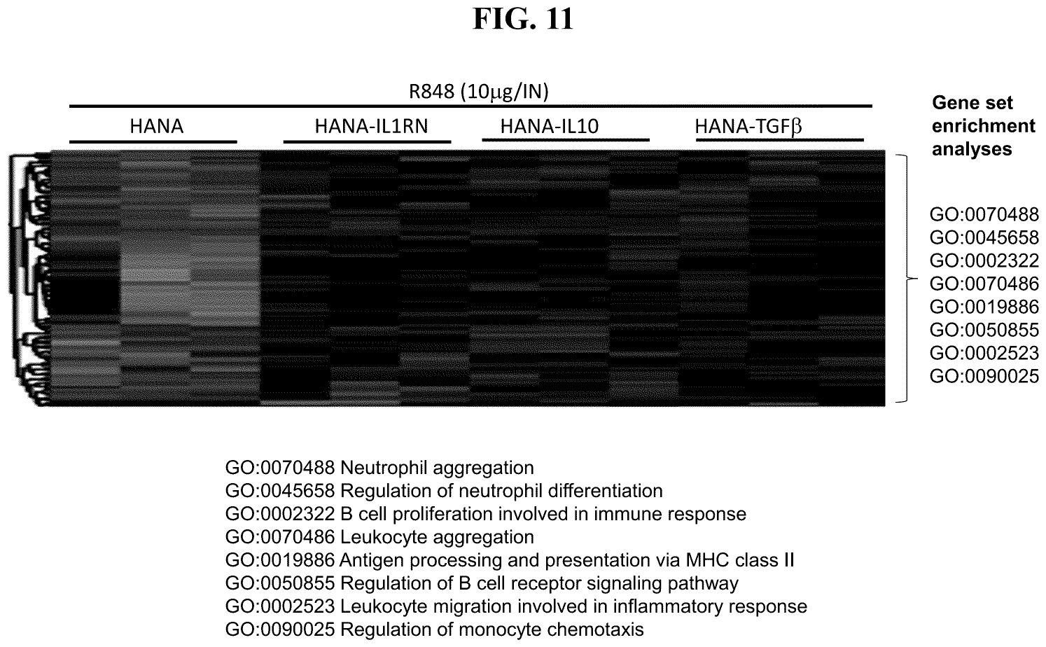

RNA-Seq analysis was performed to analyze gene expression patterns in lungs from the mice treated with HA/NA miRNA targeted AIR vector alone, or the same vector but expressing ILIRN, IL 10, or TGF-β as described above. To assess functionality of HANA-delivered cytokines in vivo, the vectors were administered to mice one day following TLR7 ligand activation with R848 (intranasal). 48 hours post R848 treatment, lungs were isolated and assessed by bulk RNA sequencing. RNA-Seq data (FIG. 11) showed that delivery of ILIRN, IL 10, or TGF-β significantly diminished the infiltration of neutrophils, T cells, and B cells in treated mice as anticipated.

1. A recombinant attenuated orthomyxovirus-based replicon (AOR) vector, wherein the AOR vector comprises vRNA segments collectively encoding, (a) optionally a biological cargo, and (b) orthomyxovirus polypeptides, or functional mutants or derivatives thereof, essential for AOR vector cell entry, cell exit and replication, wherein one or more orthomyxovirus envelope glycoproteins or functional mutants or derivatives thereof may be optionally replaced by one or more heterologous envelope glycoproteins which are capable of mediating AOR vector cell entry and/or exit, wherein one or more of the vRNA segments encoding the one or more orthomyxovirus envelope glycoproteins or functional mutants or derivatives thereof or the one or more heterologous envelope glycoproteins comprise one or more microRNA (miRNA) Silencing Elements (MSEs) such that said one or more orthomyxovirus envelope glycoproteins or functional mutants or derivatives thereof or said one or more heterologous envelope glycoproteins are not expressed or are expressed at a substantially lower level in a first cell type or species, but are expressed in a second cell type or species.

2-12. (canceled)

13. A pharmaceutical composition comprising the AOR vector of claim 1 and a pharmaceutically acceptable carrier or excipient.

14. A method for producing a biological cargo in cells of a subject in need thereof, the method comprising administering to the subject an effective amount of the AOR vector of claim 1, wherein the subject belongs to the first species or the cells belong to the first cell type.

15. A method for preventing or treating a disease in a subject in need thereof, the method comprising administering to the subject an effective amount of the AOR vector of claim 1, wherein the subject belongs to the first species.

16-19. (canceled)

20. A method of producing the AOR vector of claim 1, the method comprising introducing into a cell of the second cell type or species one or more polynucleotides encoding said vRNA segments of the AOR vector and incubating the cell under conditions suitable for AOR vector propagation.

21. The AOR vector of claim 1, wherein said AOR vector is a recombinant attenuated influenza A virus (IAV)-based replicon (AIR) vector, wherein the AIR vector comprises vRNA segments collectively encoding (a) a biological cargo and (b) IAV polypeptides, or functional mutants or derivatives thereof, essential for AOR vector cell entry, cell exit and replication, wherein IAV hemagglutinin (HA) and/or neuraminidase (NA) or functional mutants or derivatives thereof may be optionally replaced by one or more heterologous envelope glycoproteins which are capable of mediating AIR vector cell entry and/or exit, and wherein the vRNA segment(s) encoding HA and/or NA or the one or more functional mutants or derivatives thereof or the one or more heterologous envelope glycoproteins comprise one or more microRNA (miRNA) Silencing Elements (MSEs) such that said HA and/or NA or the one or more functional mutants or derivatives thereof or the one or more heterologous envelope glycoproteins are not expressed or are expressed at a substantially lower level in a first cell type or species, but are expressed in a second cell type or species.

22. The AIR vector of claim 21, wherein (i) the vRNA segment(s) encoding IAV matrix protein 1 (M1) and matrix protein 2 (M2), or functional mutants or derivatives thereof; and/or (ii) the vRNA segment encoding IAV polymerase PA subunit, or a functional mutant or derivative thereof, comprise one or more microRNA (miRNA) Silencing Elements (MSEs) such that said protein(s) or the one or more functional mutants or derivatives thereof are not expressed or are expressed at a substantially lower level in a first cell type or species, but are expressed in a second cell type or species.

23. The AIR vector of claim 21, wherein said one or more MSEs is targeted by a miRNA expressed in the first cell type or species, wherein said miRNA is not expressed or is expressed at a substantially lower level in the second cell type or species.

24. (canceled)

25. The AIR vector of claim 21, wherein the second species is chicken allantois and the first species is human, and said one or more MSEs are targeted by miR-21, miR-31, miR-192, miR-93, miR-29b, or any combination thereof.

26. The AIR vector of claim 25, wherein the vRNA segment(s) encoding HA and/or NA or the one or more functional mutants or derivatives thereof or the one or more heterologous envelope glycoproteins, the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof, comprises two or more, three or more, or four or more different MSEs which are targeted by miR-21, miR-31, miR-192, miR-93, or miR-29b, or a combination thereof.

27. The AIR vector of claim 26, wherein the vRNA segment(s) encoding HA and/or NA or the one or more functional mutants or derivatives thereof or the one or more heterologous envelope glycoproteins, the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof, comprises five MSEs which are targeted by miR-21, miR-31, miR-192, miR-93, and miR-29b, positioned in any order.

28. (canceled)

29. The AOR vector of claim 1, wherein said AOR vector is a recombinant attenuated influenza A virus (IAV)-based replicon (AIR) vector, wherein the AIR vector comprises vRNA segments collectively encoding IAV polypeptides, or functional mutants or derivatives thereof, essential for AOR vector cell entry, cell exit and replication, wherein IAV hemagglutinin (HA) and/or neuraminidase (NA) or functional mutants or derivatives thereof may be optionally replaced by one or more heterologous envelope glycoproteins which are capable of mediating AIR vector cell entry and/or exit, wherein the vRNA segment(s) encoding HA and/or NA or the one or more functional mutants or derivatives thereof or the one or more heterologous envelope glycoproteins comprise one or more microRNA (miRNA) Silencing Elements (MSEs) such that said HA and/or NA or the one or more functional mutants or derivatives thereof or the one or more heterologous envelope glycoproteins are not expressed or are expressed at a substantially lower level in a first cell type or species, but are expressed in a second cell type or species; and wherein said one or more MSEs are targeted by at least one of miR-21, miR-31, miR-192, miR-29b, or any combination thereof.

30-31. (canceled)

32. The AIR vector of claim 29, wherein the vRNA segment(s) encoding HA and/or NA or the one or more functional mutants or derivatives thereof or the one or more heterologous envelope glycoproteins, the vRNA segment(s) encoding IA V matrix protein 1 (M1) and matrix protein 2 (M2), or functional mutants or derivatives thereof, and/or the vRNA segment encoding IAV polymerase PA subunit, or a functional mutant or derivative thereof, comprises five MSEs which are targeted by miR-21, miR-31, miR-192, miR-93, and miR-29b, positioned in any order.

33. (canceled)

34. The AIR vector of claim 21, wherein said one or more MSEs are located within 3′ untranslated region (3′UTR) or 5′ untranslated region (5′UTR) of the vRNA segment(s) encoding HA and/or NA or the one or more functional mutants or derivatives thereof or the one or more heterologous envelope glycoproteins, the vRNA segment(s) encoding IAV matrix protein 1 (M1) and matrix protein 2 (M2), or functional mutants or derivatives thereof, and/or the vRNA segment encoding IAV polymerase PA subunit, or a functional mutant or derivative thereof.

35-38. (canceled)

39. The AIR vector of claim 21, wherein (i) the second species is chicken allantois, and (ii) the first species is human; and/or (i) the second cell type is an embryonated chicken egg, and (ii) the first cell type is a human cell.

40. (canceled)

41. The AIR vector of claim 21, wherein the vRNA segments collectively encode
(1) PB1, PB2, PA, HA, NP, NA, M1, M2, NS1, and NS2/NEP or functional mutants or derivatives thereof; or
(2) PB1, PB2, PA, HA, NP, NA, M1, M2, and NS2/NEP or functional mutants or derivatives thereof.

42-44. (canceled)

45. The AIR vector of claim 21, wherein the heterologous envelope glycoprotein is a vesiculovirus G protein, thogotovirus G protein, or a functional mutant or derivative thereof.

46. The AIR vector of claim 45, wherein the vRNA segments collectively encode PB1, PB2, PA, thogotovirus G protein, NP, M1, M2, and NS2/NEP or functional mutants or derivatives thereof.

47. The AIR vector of claim 41, wherein
the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or a functional mutant or derivative thereof; and
(viii) vRNA segment 8 encoding the biological cargo;
wherein
a) vRNA segment 1 further encodes NS1 or a functional mutant or derivative thereof, and vRNA segment 2 further encodes NS2 or a functional mutant or derivative thereof;
b) vRNA segment 1 further encodes NS1 or a functional mutant or derivative thereof, and vRNA segment 3 further encodes NS2 or a functional mutant or derivative thereof;
c) vRNA segment 2 further encodes NS1 or a functional mutant or derivative thereof, and vRNA segment 3 further encodes NS2 or a functional mutant or derivative thereof;
d) vRNA segment 1 further encodes NS2 or a functional mutant or derivative thereof, and vRNA segment 2 further encodes NS1 or a functional mutant or derivative thereof;
e) vRNA segment 1 further encodes NS2 or a functional mutant or derivative thereof, and vRNA segment 3 further encodes NS1 or a functional mutant or derivative thereof; or
f) vRNA segment 2 further encodes NS2 or a functional mutant or derivative thereof, and vRNA segment 3 further encodes NS1 or a functional mutant or derivative thereof.

48. The AIR vector of claim 47, wherein the vRNA segments comprise:
(i) vRNA segment 1 encoding PB2 and NS1 or functional mutants or derivatives thereof;
(ii) vRNA segment 2 encoding PB1 and NS2 or functional mutants or derivatives thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or a functional mutant or derivative thereof; and
(viii) vRNA segment 8 encoding the biological cargo.

49. The AIR vector of claim 41, wherein the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding the biological cargo; and
(viii) vRNA segment 8 encoding NS1 and NS2/NEP or functional mutants or derivatives thereof;
wherein
a) vRNA segment 1 further encodes M1 or a functional mutant or derivative thereof, and vRNA segment 2 further encodes M2 or a functional mutant or derivative thereof;
b) vRNA segment 1 further encodes M1 or a functional mutant or derivative thereof, and vRNA segment 3 further encodes M2 or a functional mutant or derivative thereof;
c) vRNA segment 2 further encodes M1 or a functional mutant or derivative thereof, and vRNA segment 3 further encodes M2 or a functional mutant or derivative thereof;
d) vRNA segment 1 further encodes M2 or a functional mutant or derivative thereof, and vRNA segment 2 further encodes M1 or a functional mutant or derivative thereof;
e) vRNA segment 1 further encodes M2 or a functional mutant or derivative thereof, and vRNA segment 3 further encodes M1 or a functional mutant or derivative thereof; or
f) vRNA segment 2 further encodes M2 or a functional mutant or derivative thereof, and vRNA segment 3 further encodes M1 or a functional mutant or derivative thereof.

50. The AIR vector of claim 41, wherein the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or functional mutants or derivatives thereof; and
(viii) vRNA segment 8 encoding NS1 and NS2/NEP or functional mutants or derivatives thereof;
wherein vRNA segment 1, 2, or 3 further encodes the biological cargo.

51. The AIR vector of claim 50, wherein the vRNA segments comprise:
(i) vRNA segment 1 encoding PB2 or a functional mutant or derivative thereof;
(ii) vRNA segment 2 encoding PB1 or a functional mutant or derivative thereof;
(iii) vRNA segment 3 encoding PA or a functional mutant or derivative thereof, and the biological cargo;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or functional mutants or derivatives thereof; and
(viii) vRNA segment 8 encoding NS1 and NS2/NEP or functional mutants or derivatives thereof.

52. The AIR vector of claim 41, wherein the vRNA segments comprise:
(i) vRNA segment 1 encoding PB2 or a functional mutant or derivative thereof;
(ii) vRNA segment 2 encoding PB1 or functional mutants or derivatives thereof;
(iii) vRNA segment 3 encoding PA or a functional mutant or derivative thereof;
(iv) vRNA segment 4 encoding HA or a functional mutant or derivative thereof;
(v) vRNA segment 5 encoding NP or a functional mutant or derivative thereof;
(vi) vRNA segment 6 encoding NA or a functional mutant or derivative thereof;
(vii) vRNA segment 7 encoding M1 and M2 or functional mutants or derivatives thereof; and
(viii) vRNA segment 8 encoding NS1, the biological cargo, and NS2/NEP or functional mutants or derivatives thereof.

53. The AIR vector of any one of claim 46, wherein the vRNA segments comprise:
(i) vRNA segment 1 encoding PB2 or a functional mutant or derivative thereof;
(ii) vRNA segment 2 encoding PB1 or functional mutants or derivatives thereof;
(iii) vRNA segment 3 encoding PA or a functional mutant or derivative thereof;
(iv) vRNA segment 4 encoding thogotovirus G protein or a functional mutant or derivative thereof;
(v) vRNA segment 5 encoding NP or a functional mutant or derivative thereof;
(vi) vRNA segment 6 encoding the biological cargo;
(vii) vRNA segment 7 encoding M1 and M2 or functional mutants or derivatives thereof; and
(viii) vRNA segment 8 encoding NS1 and NS2/NEP or functional mutants or derivatives thereof.

54. The AIR vector of claim 21, wherein the biological cargo is an antigen, a biologically active polypeptide, a non-coding RNA (ncRNA), a marker polypeptide, one or more components of a gene editing system, or a Cre recombinase.

55-59. (canceled)

60. The AIR vector of claim 25, wherein said one or more MSEs comprise one or more reverse complement sequences of miR-21 comprising the sequence: UAGCUUAUCAGACUGAUGUUGA (SEQ ID NO: 1), miR-31 comprising the sequence AGGCAAGAUGCUGGCAUAGCU (SEQ ID NO: 2), miR-192 comprising the sequence CUGACCUAUGAAUUGACAGCC (SEQ ID NO: 3), miR-93 comprising the sequence CAAAGUGCUGUUCGUGCAGGUAG (SEQ ID NO: 4), or miR-29b-3p: UAGCACCAUUUGAAAUCAGUGUU (SEQ ID NO: 5), or any combination thereof.

61. (canceled)

62. A pharmaceutical composition comprising the AIR vector of claim 21 and a pharmaceutically acceptable carrier or excipient.

62. A method for producing a biological cargo in airway cells of a subject in need thereof, the method comprising administering to the subject an effective amount of the AIR vector of claim 21, wherein the subject belongs to the first species or comprises the first cell types.

63. A method for preventing or treating a disease in a subject in need thereof, the method comprising administering to the subject an effective amount of the AIR vector of claim 21, wherein the subject belongs to the first species or comprises the first cell types.

64-67. (canceled)

68. A method of producing the AIR vector of claim 21, the method comprising introducing into a cell of the second cell type or species one or more polynucleotides encoding vRNA segments of the AIR vector of claim 21 and incubating the cell under conditions suitable for AIR vector propagation.


SEQUENCE LISTING (5 sequences)

SEQ ID NO: 1 - RNA, 22 nt, synthetic construct
  source 1..22: mol_type = other RNA
tagcttatcagactgatgttga

SEQ ID NO: 2 - RNA, 21 nt, synthetic construct
  source 1..21: mol_type = other RNA
aggcaagatgctggcatagct

SEQ ID NO: 3 - RNA, 21 nt, synthetic construct
  source 1..21: mol_type = other RNA
ctgacctatgaattgacagcc

SEQ ID NO: 4 - RNA, 23 nt, synthetic construct
  source 1..23: mol_type = other RNA
caaagtgctgttcgtgcaggtag

SEQ ID NO: 5 - RNA, 23 nt, synthetic construct
  source 1..23: mol_type = other RNA
tagcaccatttgaaatcagtgtt